(19) United States
(12) Patent Application Publication
(10) Pub. No.: US 2026/0272656 A1
(43) Pub. Date: Sep. 17, 2026

(54) FLOW RESTRICTION DEVICES WITH COVERED PORTIONS

(71) Applicant: Edwards Lifesciences Corporation, Irvine, CA (US)

(72) Inventors: Aviran Pitusi, Rosh Haayin (IL); Amir Davidesko, Binyamina Givat Ada (IL); Aviv Geva, Hod Hasharon (IL); Anatoly Dvorsky, Haifa (IL); Danny M. Garmahi, Hadera (IL)

(21) Appl. No.: 19/670,996

(22) Filed: May 7, 2026

Related U.S. Application Data
(63) Continuation of application No. PCT/US2024/056539, filed on Nov. 19, 2024.
(60) Provisional application No. 63/604,807, filed on Nov. 30, 2023.

(51) Int. Cl. A61F 2/24; A61F 2/07; A61F 2/48; A61B 5/021; A61F 2/06; A61F 2/90

(52) U.S. Cl. CPC A61F 2/2475; A61F 2/07; A61F 2/2418; A61F 2/482; A61B 5/021; A61F 2002/068; A61F 2/90; A61F 2210/0076; A61F 2230/005; A61F 2250/001; A61F 2250/0018

(57) ABSTRACT

An implantable device is for dynamically occluding a blood vessel is described. The implantable device may include an annular frame having a lumen extending therethrough. A sealing element can be disposed at least along an exterior of the frame. The sealing member may take the form of a polymeric covering. An actuation member is provided for altering a diameter of the frame to modulate the flow of blood through the lumen. The actuation member may be adapted to alter the diameter of the frame based on a signal from a processor. The processor can receive a signal from a pressure sensor. A power source may be provided for actuating the actuation element.

### CROSS-REFERENCE TO RELATED APPLICATIONS

This application is a continuation of PCT Application No. PCT/US2024/056539, filed Nov. 19, 2024, which claims the benefit of U.S. Provisional Patent Application No. 63/604,807, filed Nov. 30, 2023, each of which is hereby incorporated by reference in its entirety.

### BACKGROUND

This disclosure relates generally to the field of medical devices and procedures, and more specifically to the field of blood flow management in blood vessels.

Chronic kidney disease (CKD) is a common comorbidity with many patients who suffer from Heart Failure (HF). HF patients may also experience impaired renal function because impaired renal function can be caused by increased systemic venous congestion as a result of low cardiac output and/or low blood pressure. Conventional medical devices for relieving pressure may include static balloons that radially inhabit a portion of a blood vessel when implanted.

### SUMMARY

Described herein are one or more methods and/or devices to facilitate management of blood flow through and/or into one or more blood vessels and/or chambers of a heart. There is a need for new and useful system and method for modulating blood flow through a blood vessel. In particular, the methods and devices described herein may manage blood flow through a blood vessel using flow restriction mechanisms that are shaped in orientations other than radial ended flow restrictors.

In some examples, the systems and methods described herein may function to limit central venous volume to operate in a bi-modal fashion that allows device configuration to reduce venous pressure when the patient is at rest, yet allows undisturbed or minimally disturbed venous flow when the patient exercises, so as to meet the dynamic blood flow and/or blood pressure requirements of the patient.

In some aspects, the techniques described herein relate to an implantable device for modulating blood flow through a blood vessel. The implantable device may include a frame having an inflow end, an actuatable outflow end, and a longitudinal axis extending therethrough, the frame including: an inner wall with a first polymeric covering: an outer wall with a second polymeric covering, wherein the first polymeric covering is coupled to portions of the second polymeric covering to form a circumferentially sealed space defined to extend around a portion of a circumference of the outer wall of the frame; and a skirt membrane wrapped around an exterior surface of the second polymeric covering and disposed offset from a lateral centerline at a midpoint of the frame and toward the inflow end of the frame.

In some aspects, the techniques described herein relate to an implantable device for modulating blood flow through a blood vessel, the implantable device including: a frame having a proximal end and a distal end with a longitudinal axis extending therethrough, the frame including: an inner surface having a first polymeric covering; an outer surface having a second polymeric covering, wherein the first polymeric covering is coupled to portions of the second polymeric covering to form a circumferentially sealed space extending along at least a portion of frame, the circumferentially sealed space housing at least one waist member extending around an outer circumference of the outer surface and encased in a space between the first polymeric covering and the second polymeric covering; and an actuation member coupled to the frame and configured to actuate the at least one waist member to collapse the distal end of the frame toward a central axis of the frame, or expand the distal end of the frame away from the central axis, in response to an actuation of a control wire coupled to the at least one waist member.

In some aspects, the techniques described herein relate to a method of treatment for reducing blood flow at a target site in a blood vessel of a heart of a subject, the method including: introducing a device in the blood vessel, the device including: a frame having an inflow end, an actuatable outflow end, and a longitudinal axis extending therethrough, the frame including: an inner wall with a first polymeric covering: an outer wall with a second polymeric covering, wherein the first polymeric covering is coupled to portions of the second polymeric covering to form a circumferentially sealed space defined to extend around a portion of a circumference of the outer wall of the frame; and a skirt membrane wrapped around an exterior surface of the second polymeric covering and disposed offset from a lateral centerline at a midpoint of the frame and toward the inflow end of the frame; and actuating the device to modulate a flow of blood within the blood vessel.

In some aspects, the techniques described herein relate to an implantable device for dynamically occluding a blood vessel, the implantable device including: a tubular frame having a lumen extended therethrough; a sealing element disposed along an exterior of the frame and along an interior of the frame opposite the exterior of the frame, the sealing element being configured to seal portions of the frame between the exterior and the interior of the frame; and an actuation member coupled to the frame and configured to alter a diameter of the frame to modulate blood flow through the lumen.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1A is a flat view of an example frame of a device for modulating blood flow through a blood vessel.

FIG. 1B is a flat view of FIG. 1A depicting example apertures for use in controlling modulation of blood flow through a blood vessel.

FIG. 1C is a side view of the frame of FIG. 1A that includes an actuator.

FIG. 1D is a perspective view of the frame of FIG. 1C depicting example polymeric coverings.

FIG. 1E is a side view of the frame of FIG. 1D including a plurality of eyelets for receiving an actuatable control wire.

FIG. 1F is a side view of the frame of FIG. 1E in an at least partially collapsed position.

FIG. 1G is a side view of the frame of FIG. 1E and a zoomed in view of a waist member installed therein.

FIG. 1H is a side view of a frame depicting an example configuration of rings and detents.

FIG. 1I is a block diagram depicting an example configuration of rings and detents.

FIG. 1J is another example configuration of rings and detents for the frame of FIG. 1E.

FIG. 1K is a side view of a frame depicting an example configuration of rings.

FIG. 1L is an example configuration of rings.

FIG. 1M is another example configuration of rings.

FIG. 2A is an example cross-sectional view of an outflow end of the devices described herein in a restricted blood flow state.

FIG. 2B is an example cross-sectional view of an outflow end of the devices described herein in a partially restricted blood flow state.

FIG. 2C is another example cross-sectional view of an outflow end of the devices described herein in a partially restricted blood flow state.

FIG. 2D is an example cross-sectional view of an outflow end of the devices described herein in an unrestricted blood flow state.

FIG. 3 is an example perspective view of a flow restricting device and an example delivery system for deploying the flow restricting device in a blood vessel.

FIG. 4 illustrates a block diagram of an example system for modulating blood flow through one or more blood vessels.

FIG. 5A illustrates a schematic diagram of an example embodiment of a system for modulating blood flow through a blood vessel.

FIG. 5B illustrates a schematic diagram of an example embodiment of a system for modulating blood flow through a blood vessel.

FIG. 6 illustrates a flow diagram of an example process of modulating blood flow through one or more blood vessels.

FIG. 7A illustrates a schematic representation of portions of a human subject in which the devices described herein may be implanted.

FIG. 7B illustrates a schematic representation of a portion of a human subject with a device described herein implanted.

FIG. 8A illustrates example radial force plot curves measured during expanding and crimping processes occurring over time for the devices described herein without covered portions.

FIG. 8B illustrates example radial force plot curves measured during expanding and crimping processes occurring over time for the devices described herein with covered portions.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure and protection to these embodiments. Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

In general, the systems and methods described herein may enable modulating and/or balancing of blood flow through a blood vessel. The modulating and/or balancing of blood flow may be performed by the devices described herein to occlude, partially occlude, and/or otherwise manage or regulate blood flow to or through a portion of a blood vessel. In some examples, such modulation and/or balancing of blood flow to or through a blood vessel may result in additionally modulating pressure in the right atrium of the heart and/or other organs of the body.

The examples presented herein may relate to providing devices, methods, and/or methods of treatment (MOTs) for modulating, regulating and/or otherwise managing blood flow to or through one or more blood vessels. The terminology of restricting blood flow, regulating blood flow, modulating blood flow, managing blood flow, and balancing blood flow cause regulation of blood pressure, modulation of blood pressure, management of blood pressure, and/or balancing of blood pressure. As such, for example, a flow modulation device is synonymous with a pressure regulating device (i.e., a flow regulator is synonymous with a pressure regulator). In some examples, the devices described herein may include blood flow management devices for reducing blood flow through a blood vessel, such as a vena cava, a Superior Vena Cava (SVC) and an Inferior Vena Cava (IVC), or related vessels. Managing blood flow through the SVC or IVC can be achieved by the devices described herein to provide an advantage of improving perfusion of the kidneys. In particular, the devices described herein may generate a pressure gradient across the kidneys by decreasing central venous pressure by restricting, balancing, or otherwise modifying blood flow through the SVC and/or IVC, resulting in improved kidney perfusion and function.

In some examples, the devices, methods, and/or MOTs described herein may be utilized to solve a technical problem of unwanted pressure increases in the right atrium in patients that have chronic kidney disease (CKD) and/or congestive heart failure (CHF). For example, patients with CKD and/or CHF may exhibit reduced kidney function when pressure in the right atrium of the heart is above a predefined pressure threshold. The predefined pressure threshold may be used as a basis to determine whether a patient is exhibiting low vessel pressure (e.g., below the predefined pressure threshold) or high vessel pressure (e.g., above the predefined pressure threshold). When vessel pressure is determined to be high, the devices, methods, and/or MOTs can provide a technical solution to the technical problem recited above. For example, each of the devices described herein may be used to decrease pressure within one or more vessels to avoid right atrium pressure increases and/or pressure variations. In particular, the devices, methods, and/or MOTs described herein can be used to reduce and maintain low pressure in the right atrium, which provides a technical effect of enabling the kidneys to more effectively filter blood.

In some examples, in CHF and/or CKD patients, a renal pressure gradient (between the renal arteries and renal veins) may be decreased due to elevated renal venous pressure thereby lowering glomerular filtration rate (GFR), which represents the rate at which the kidney filters blood. One example rate indicative of CKD may include a GFR below, for example, about 90 mL/minute. In general, reduction of renal venous pressure may improve GFR levels and reduce blood volume retention. However, it may be desirable for a device that limits central venous volume to operate in a bi-modal fashion to reduce venous pressure when the patient is at rest, yet allow undisturbed or minimally disturbed venous flow when the patient exercises, so as to meet the dynamic flow/pressure requirements. Moreover, it may be desired for any such solution, when provided as an implantable device, to be percutaneously deliverable and to operate in a manner that minimizes risk of thrombosis. Some conventional devices may utilize flow restrictor elements (e.g., balloons) that protrude radially inward within a blood vessel to restrict or limit blood through the implanted device. A potential drawback with such conventional solutions relates to flow disturbances that may result from the radial orientation of the restricting elements. Thus, the devices, methods, and/or MOTs described herein may be an active stent device implanted in a blood vessel and functionable to cinch an end portion of the stent toward and away from a centerline of the stent to modulate blood flow through a blood vessel. The example stent devices described herein can perform blood flow management actively and/or passively to assist in reducing and/or maintaining right atrium pressures to a relatively low pressure even when a surge in blood volume occurs in one or more vessels of the venous system.

### Systems and Devices

Disclosed herein are systems and methods for modulating blood flow through a blood vessel. In some examples, the implantable flow modulating devices described herein may be used in blood flow occlusion therapy. For example, the devices described herein may relate to venous occlusion therapy using implantable and/or electronically controlled flow restricting devices for the treatment of acute heart failure. Some devices may be non-implantable or partially implantable. In some examples, the devices described herein generally function to occlude or partially occlude a blood vessel, such as the SVC, the IVC, and/or other vessels or junctions (e.g., a vena cava, an azygos junction, etc.). In such examples, blocking or occluding such junctions or other vessels can ensure that blood may not bypass the devices described herein, but instead flow through an inner diameter of such devices. In some examples, the devices described herein have been contemplated for use in a patient/user having chronic heart failure and/or chronic kidney disease, but may be used in any vessel for flow regulation therethrough.

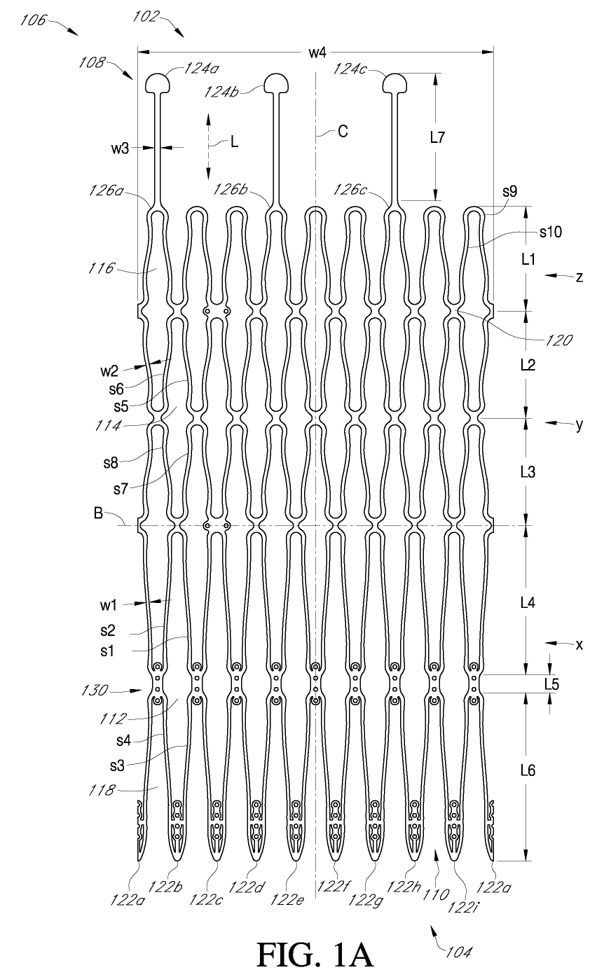

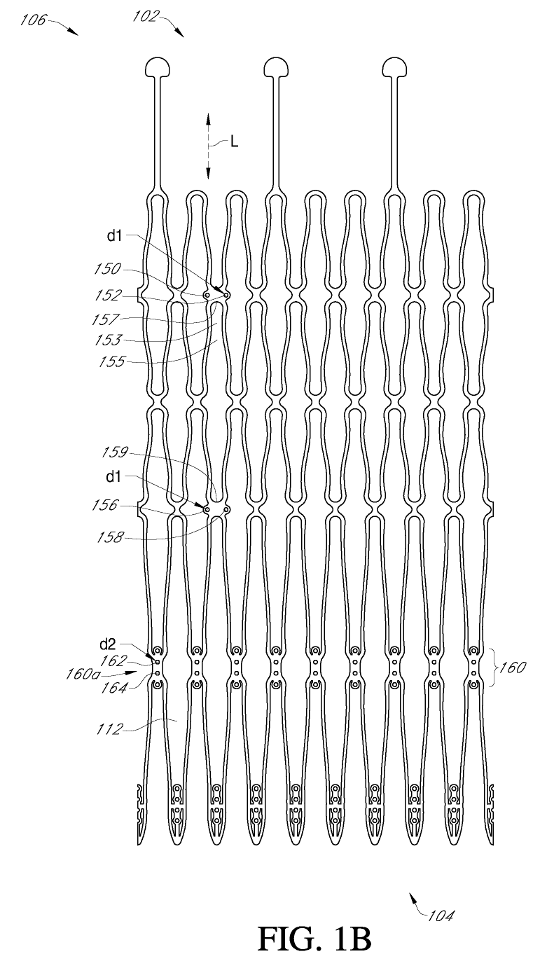

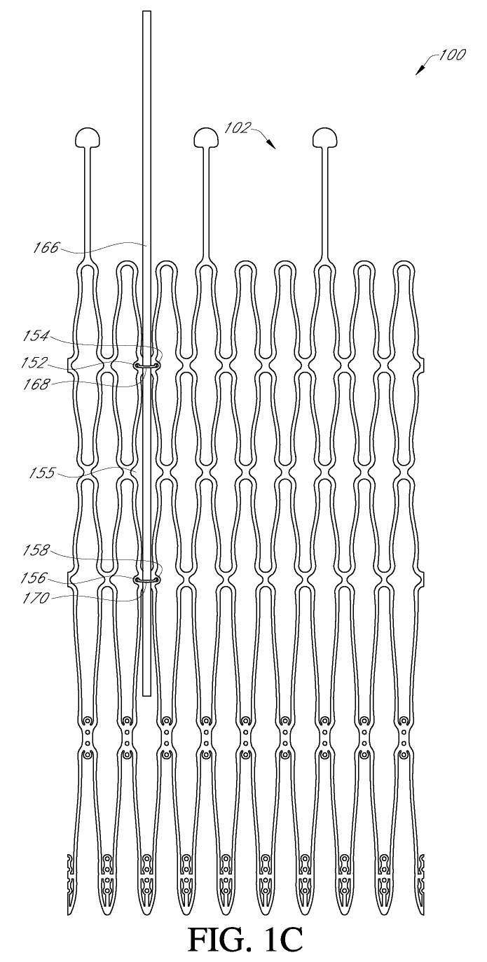

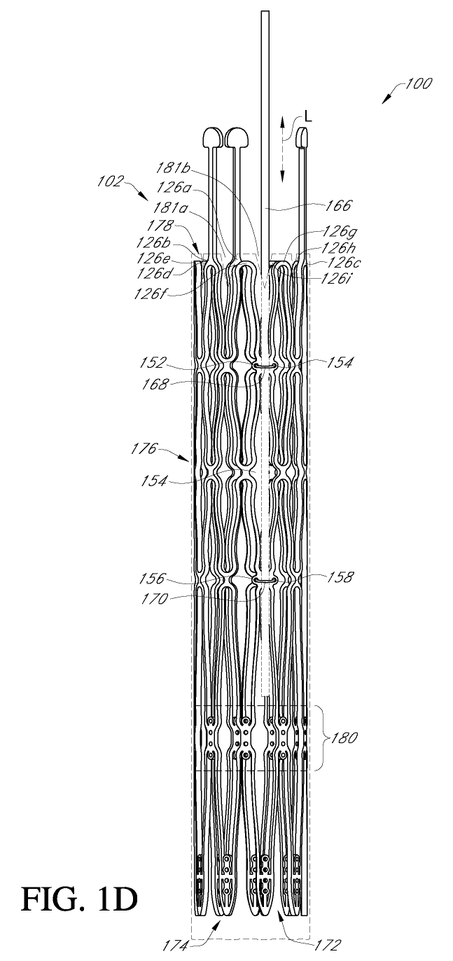

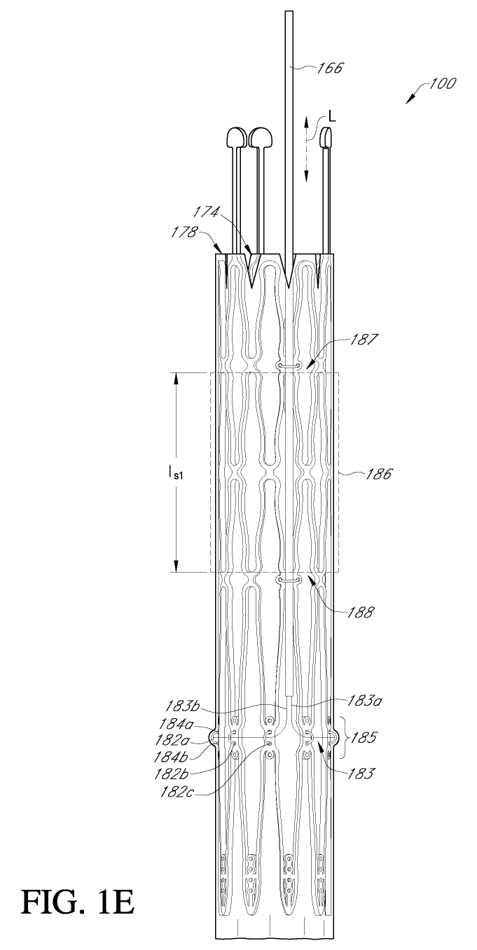

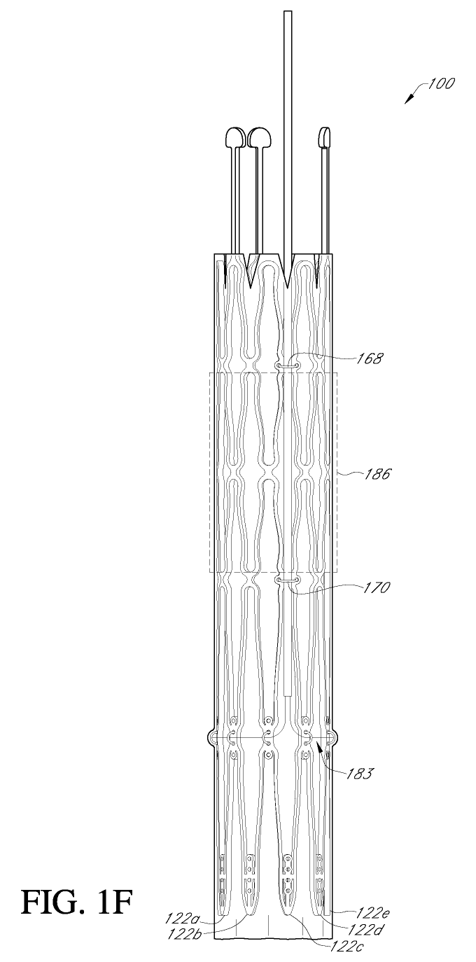

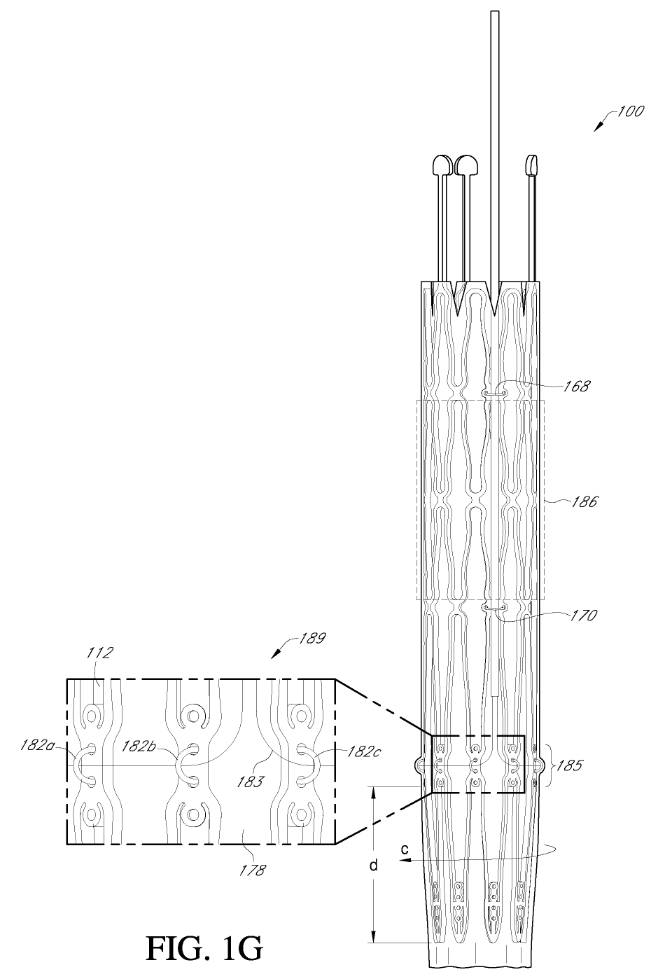

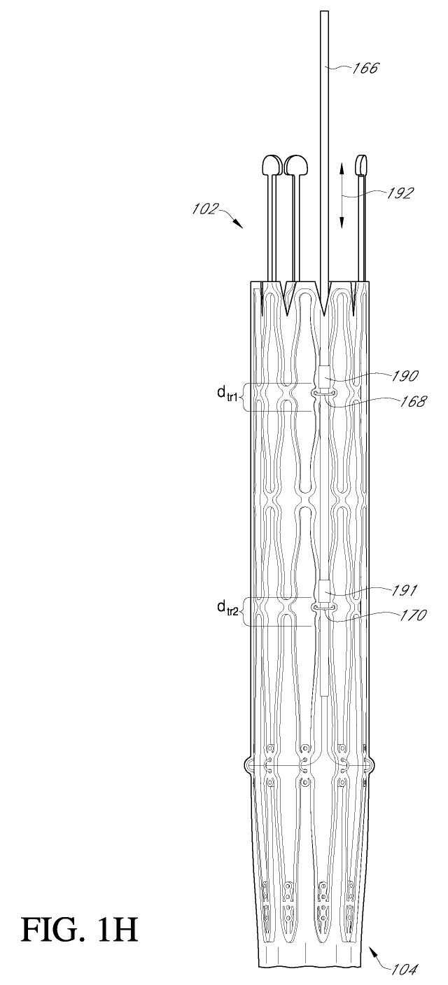

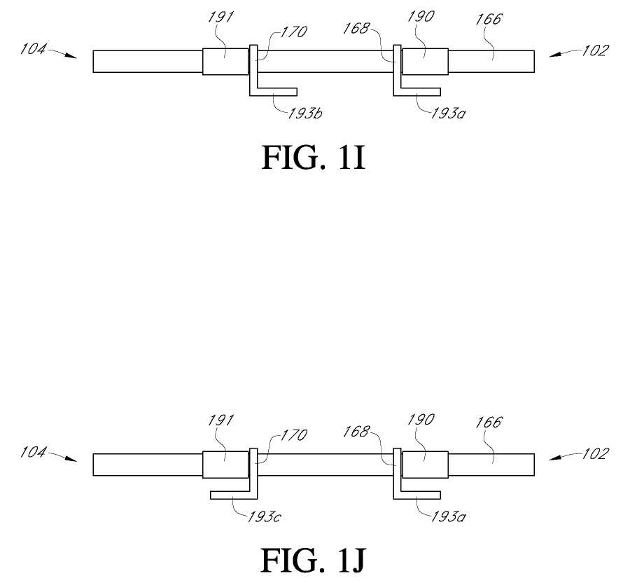

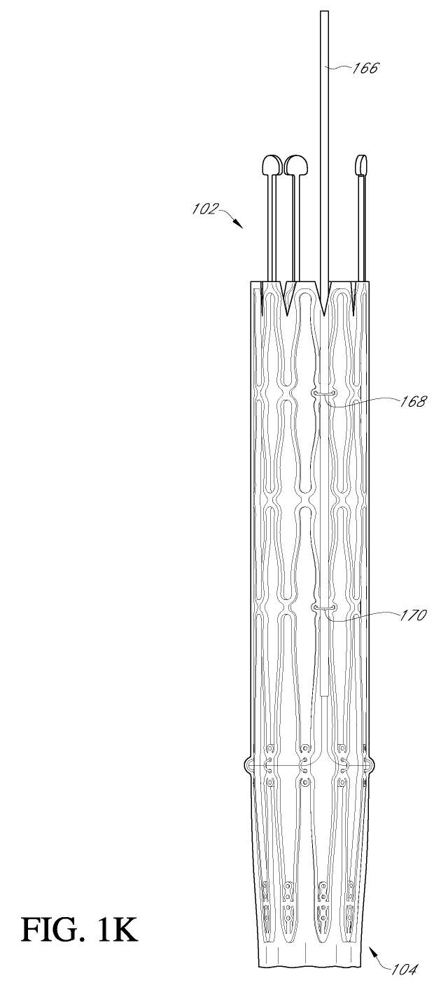

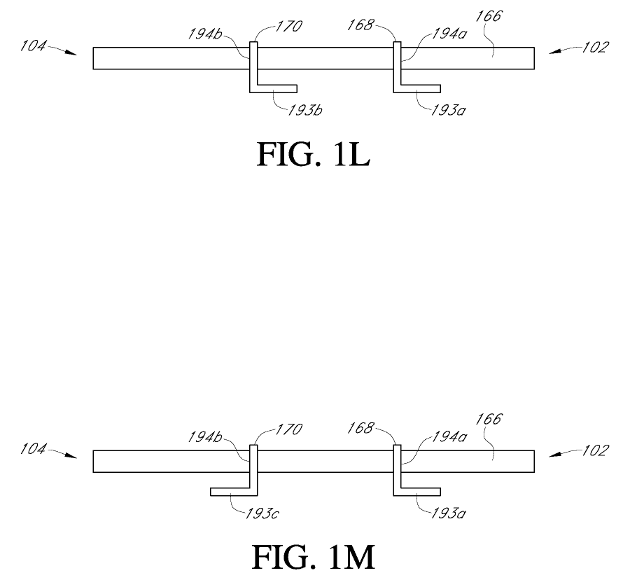

FIGS. 1A-1M illustrate views of an example flow modulating device 100 (e.g., FIG. 1C) for modulating blood flow through a blood vessel. The device 100 may be implanted into a blood vessel, such as the superior vena cava, the inferior vena cava, or any other blood vessel where modulating blood flow is desired. For example, the device 100 may modulate a volume of blood flowing from the superior vena cava into a right atrium to decrease right atrial pressure.

The device 100 may include a self-expanding frame (e.g., stent, braid, etc.) that may be delivered into the blood vessel (e.g., via jugular access, subclavian access, or transfemoral access) using a sheathed catheter (not shown). In some examples, the frame may be partially or fully encased in at least one polymeric layer. In some examples, the device 100 includes one or more controls for expanding and constricting (uniformly or nonuniformly) a perimeter of an end portion of the frame. In some examples, the device 100 further includes a skirt membrane wrapped around at least a portion of an exterior surface of the frame.

FIG. 1A is a flat view of an example frame 106 that may be included in device 100 to modulate blood flow through a blood vessel. In this example, the frame 106 is unrolled from a cylindrical shape and may be assembled about central axis (C), as shown in FIG. 1B, for example. The flat view is depicted for ease of viewing the features of the frame 106.

The frame 106 may be expandable and/or contractable and includes a proximal end 108 and a distal end 110, and a longitudinal axis (L) extending therethrough. As used herein, the central axis (C) of the expandable frame 106 is substantially parallel to the longitudinal axis (L). The proximal end 108 may correspond to an inflow end 102 of the device 100. Similarly, the distal end 110 may correspond to an outflow end 104 of the device 100. In some examples, the outflow end 104 is an actuatable end that may expand and constrict (uniformly or nonuniformly) to open, partially close, or close the outflow end 104 of the device 100.

The frame 106 may be a stent (or braid) constructed of metal wire (e.g., stainless steel, platinum, Nitinol® wire or another shape memory alloy), or other material suitable for implantation in the human body. In some examples, the frame 106 is a bare metal stent, such that the frame 106 may be arranged to be at least partially incorporated into an inner wall of the blood vessel. In some examples, the frame 106 includes one or more additional coverings such as sleeves, skirts, etc., as described elsewhere herein.

As shown in FIG. 1A, the frame 106 is formed from a plurality of struts (e.g., strut s1, strut s2, strut s3, etc.) that form cells (e.g., cell 116, cell 112, etc.) spaced from the inflow end 102 to the outflow end 104 and extending from each other around the frame 106. For example, the struts may form two or more rows (e.g., a first row (x), a second row (y), a third row (z), etc.) of cells (e.g., cell 112, cell 114, cell 116, partial cell 118, etc.). In some examples, the struts may form one or more rings of cells. The rings may be stacked from the inflow end 102 to the outflow end 104. For example, a number of adjacent rows of cells formed of struts (e.g., diamond-shaped cells, polygon-shaped cells, etc.) may form the rings. For example, the frame 106 may include one or more rings of cells stacked from the inflow end 102 to the outflow end 104.

In one non-limiting example, the cell structures may be formed by (1) at least two struts arranged at a first strut angle to form a first arched crown adjacent the inflow end 102 (2) at least two struts arranged at the first strut angle to form a second arched crown opposite the first arched crown and toward the outflow end of the frame 106. The remainder of the cell shape may be formed at a central and lateral bisect (B) of the shaped cell structure by a connector portion 120 that connects the second arched crown to a third arched crown belonging to an adjacent cell on a first side and an adjacent cell on a second side where the first side is opposite the second side.

The first row (x) may include struts that are longer in length and thinner and/or tapered in width than the struts in the second row (y) or third row (z) resulting in cells (e.g., cell 112) of the first row (x) being elongated to minimize radial force at the outflow end (e.g., distal end 110). The thinner, tapered struts may allow for resilient bending of outflow crowns 122a, 122b, 122c, 122d, 122e, 122f, 122g, 122h, and 122i toward central axis (C) to place the device 100 in a restricted or unrestricted blood flow state.

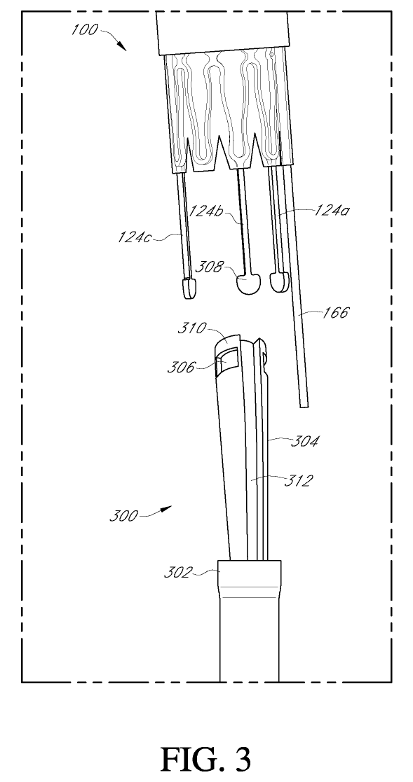

As shown, the first row (x) may terminate in a plurality of crowns 122a-122i, which can be caused to move radially inward toward a central axis (C) of the frame 106 and move radially outward away from the central axis (C). The second row (y) may extend from the first row (x) longitudinally toward the proximal end (e.g., inflow end 102) of the frame 106. The third row (z) may include (or terminate in) one or more deployment members such as deployment member 124a, deployment member 124b, and/or deployment member 124c. Each deployment member 124a, 124b, 124c may be coupled to (or be an extension of) a respective crown (e.g., crown 126a, crown 126b, and/or crown 126c). Each deployment member 124a, 124b, 124c may have a bulbous end for being received in an accommodation cavity of a delivery device (e.g., delivery system 300 of FIG. 3). Each deployment member may be housed in system 300 until being released to cause implantation of the implantable device 100 into a blood vessel.

In some examples, the first row (x) is arranged at the distal end 110 (e.g., outflow end 104) of the frame 106. The first row (x) may include a plurality of cells (e.g., 112) with an elongated diamond shape. In addition, the first row (x) may terminate at the outflow end 104 in crowns 122a-122i, each separated by a space (e.g., partial cell 118). Each cell (e.g., cell 112) may be formed of a first plurality of struts (e.g., s1, s2) of a first length (L4) and first width (W1) and a second plurality of struts (e.g., s2, s4) with either the same length (L4) or an extended length (L6) that is about 2 percent to about 15 percent longer than the length (L4); about 2 percent to about 4 percent longer than the length (L4); about 4 percent to about 6 percent longer than the length (L4); about 6 percent to about 8 percent longer than the length (L4); about 8 percent to about 10 percent longer than the length (L4); about 10 percent to about 12 percent longer than the length (L4); about 12 percent to about 14 percent longer than the length (L4); or about 14 percent to about 15 percent longer than the length (L4).

The extended length (L6) may account for modifications to a shape and size of each crown 122a-122i to account for expanding or contracting the outflow end 104 of the frame 106 (i.e., an outflow end of device 100). The first plurality of struts (e.g., s1,s2) may be coupled to the second plurality of struts (e.g., s2, s4) by a center portion (e.g., center portion 130) of the cells (e.g., cell 112), which may function as a connector between strut s1 and strut s3 and between strut s2 and strut s4.

In some examples, the second row (y) may include cells (e.g., cell 114) with an elongated diamond shape formed of a second plurality of struts (e.g., s5, s6, s7, and s8). The struts s5, s6, s7, and s8 may have a second length (L2) or a length (L3). In some examples, length (L2) is equal to length (L3). In some examples, length (L2) is greater than length (L3). In some examples, length (L2) is less than length (L3). The struts s5, s6, s7, and s8 may have a second width (w2) that is thicker or larger than width (w1) to ensure a higher radial force (e.g., stiffness) in row (y) as compared to the first row (x). That is, the first length (L4) may be greater than the second length (L3) and/or (L2) and the first width (w1) may be less than the second width (w2).

The third row (z) may extend longitudinally from the second row (y) and to the proximal end (e.g., inflow end 102). The third row (z) may be formed of a third plurality of struts (e.g., strut s9, strut s10) of a third length (L1). The third row (z) may couple to the second row (y) by a connector (e.g., connector 120).

The frame 106 may have a length from the outflow end 104 to an end of a deployment member (e.g., deployment member 124a) of about 45 mm to about 75 mm; about 45 mm to about 50 mm; about 50 mm to about 55 mm; about 55 mm to about 60 mm; about 60 mm to about 65 mm; about 65 mm to about 70 mm; or about 70 mm to about 75 mm.

The frame 106 may have a width (w4) in a flat orientation of about 20 mm to about 35 mm; about 20 mm to about 22 mm; about 22 mm to about 25 mm; about 25 mm to about 28 mm; about 28 mm to about 30 mm; about 30 mm to about 33 mm; or about 33 mm to about 35 mm.

The frame 106 may have a diameter of about 3 mm to about 6 mm; about 3 mm to about 3.2 mm; about 3.2 mm to about 3.6 mm; about 3.6 mm to about 3.9 mm; about 3.9 mm to about 4.2 mm; about 4.2 mm to about 4.5 mm; about 4.5 mm to about 4.8 mm; about 4.8 mm to about 5.1 mm; about 5.1 mm to about 5.3 mm; about 5.3 mm to about 5.6 mm; or about 5.6 mm to about 6 mm.

The frame covered by one or more layers of polymer that may further extend a length of the frame 106 on the inflow end 102 and/or the outflow end 104 by an additional length of about 0.1 mm to about 5 mm; about 0.1 mm to about 0.3 mm; about 0.3 mm to about 0.5 mm; about 0.5 mm to about 1 mm; about 1 mm to about 1.5 mm; about 1.5 mm to about 3 mm; about 3 mm to about 4 mm; or about 4 mm to about 5 mm.

The length (L1) may be about 6 mm to about 10 mm; about 6 mm to about 6.5 mm; about 6.5 mm to about 7 mm; about 7 mm to about 7.5 mm; about 7.5 mm to about 8 mm; about 8 mm to about 8.5 mm; about 8.5 mm to about 9 mm; about 9 mm to about 9.5 mm; or about 9.5 mm to about 10 mm.

Length (L2) may be about 6 mm to about 10 mm; about 6 mm to about 6.5 mm; about 6.5 mm to about 7 mm; about 7 mm to about 7.5 mm; about 7.5 mm to about 8 mm; about 8 mm to about 8.5 mm; about 8.5 mm to about 9 mm; about 9 mm to about 9.5 mm; or about 9.5 mm to about 10 mm.

Length (L3) may be about 6 mm to about 10 mm; about 6 mm to about 6.5 mm; about 6.5 mm to about 7 mm; about 7 mm to about 7.5 mm; about 7.5 mm to about 8 mm; about 8 mm to about 8.5 mm; about 8.5 mm to about 9 mm; about 9 mm to about 9.5 mm; or about 9.5 mm to about 10 mm.

Length (L4) may be about 10 mm to about 15 mm; about 10 mm to about 11 mm; about 11 mm to about 12 mm; about 12 mm to about 13 mm; about 13 mm to about 14 mm; or about 14 mm to about 15 mm.

Length (L5) may be about 2 mm to about 5 mm; about 2mm to about 2.3 mm; about 2.3 mm; about 2.6 mm to about 2.9 mm; about 2.9 mm to about 3.2 mm; about 3.2 mm to about 3.5 mm; about 3.5 mm to about 3.8 mm; or about 3.8 mm to about 4.1 mm; about 4.1 mm to about 4.3 mm; about 4.3 mm to about 4.6 mm; or about 4.6 mm to about 5 mm.

Length (L6) may be about 10 mm to about 16 mm; about 10 mm to about 11 mm; about 11 mm to about 12 mm; about 12 mm to about 13 mm; about 13 mm to about 14 mm; about 14 mm to about 15 mm; about 15 mm to about 16 mm.

Length (L7) may be about 9 mm to about 14 mm; about 9 mm to about 10 mm; about 10 mm to about 11 mm; about 11 mm to about 12 mm; about 12 mm to about 13 mm; or about 13 mm to about 14 mm.

Width (w1) may represent a width of one or more struts in row (x). Width (w1) may be about 0.1 mm to about 0.3 mm; about 0.1 mm to about 0.15 mm; about 0.15 mm to about 0.2 mm; about 0.2 mm to about 0.25 mm; or about 0.25 mm to about 0.3 mm.

Width (w2) may represent a width of one or more struts in row (y). Width (w2) may be about 0.2 mm to about 0.35 mm; about 0.2 mm about 0.22 mm; about 0.22 mm to about 0.24 mm; about 0.24 mm to about 0.26 mm; about 0.26 mm to about 0.29 mm; or about 0.29 mm to about 0.3 mm to about 0.35 mm.

Width (w3) may represent a width of one or more struts in row (z). Width (w3) may be about 0.4 mm to about 0.6 mm; about 0.45 mm to about 0.5 mm; about 0.5 mm to about 0.55 mm; or about 0.55 mm to about 0.6 mm.

In general, the struts in the first row (x), second row (y), and third row (z) may have a variable radial stiffness along the circumference of the device 100 from the proximal end 102 to the distal end 104. For example, struts in the first row (x) may be of a particular width to provide a first radial stiffness against the vessel. The struts of the second row (y) may have a particular width to provide a second radial stiffness. In general, the first radial stiffness may be less than the second radial stiffness to allow for improved bending of struts in the first row (x).

FIG. 1B is a flat view of FIG. 1A depicting example apertures for use in controlling modulation of blood flow through a blood vessel. In some examples, the apertures described herein may be used to couple the frame 106 to actuation members and/or related components. For example, a first set of apertures includes an aperture 150 and an aperture 152. Apertures 150, 152 are arranged adjacent to an apex 157 of an arched crown 153 of a cell 155. As shown, the aperture 150 may be in line with aperture 152 along a plane perpendicular to the longitudinal axis (L) of the frame 106. A second set of apertures includes an aperture 156 and an aperture 158. Apertures 156, 158 are arranged at a base 159 of an arched crown of the cell 155. Aperture 156 is in line with aperture 158 along a plane perpendicular to the longitudinal axis (L) of the frame 106. As shown, the first set of apertures (e.g., apertures 150, 152) are arranged along the same azimuth as the second set of apertures (e.g., apertures 156, 158) and parallel to the longitudinal axis of the frame 106.

Each aperture 150, 152, 156, and 158 may have a diameter (d1) of about 0.25 mm to about 0.4 mm; about 0.25 mm to about 0.28 mm; about 0.28 mm to about 0.3 mm; about 0.3 mm to about 0.35 mm; or about 0.35 mm to about 0.4 mm. Each aperture 150, 152, 156, and 158 may be coupled to one or more connecting elements (see FIG. 1C, element 168, element 170) including, but not limited to, one or more rings, eyelets, u-shaped connectors, brackets, or the like and are described in further detail in at least FIG. 1C.

The frame 106 may further include a plurality of joints 160 in row (x). Each cell (e.g., cell 112, etc.) in the first row (x) includes a joint (e.g., joint 160a) at a midpoint of the cell 112. Each joint 160 (e.g., joint 160a) includes a first aperture (e.g., aperture 162) and a second aperture (e.g., aperture 164). The first aperture 162 and the second aperture 164 may be aligned along the longitudinal axis (L) of the frame 106. Each aperture 162, 164 may have a diameter (d2) of about 0.25 mm to about 0.4 mm; about 0.25 mm to about 0.28 mm; about 0.28 mm to about 0.3 mm; about 0.3 mm to about 0.35 mm; or about 0.35 mm to about 0.4 mm.

Each joint 160 may be arranged to receive one or more connecting elements (e.g., eyelets, rings, supports, brackets, etc.) that may form a line of apertures that may receive a wire, thread, or other length of material that may be used to cinch a portion of the frame 106 to cause the outflow crowns 122a-122i to collapse inward toward central axis (C) or expand outward away from central axis (C), as described elsewhere herein.

FIG. 1C is a side view of the frame of FIG. 1A that includes an actuation member 166. The actuation member 166 may be an elongate tube arranged substantially parallel to the longitudinal axis (L). The actuation member 166 may extend beyond the inflow end 102 of the frame 106 and may be actuated to modify the flow of blood through a blood vessel in which device 100 is implanted. The actuation member 166 may be shaped to hold a control wire (e.g., control wire 183) threaded through the elongate tube as described elsewhere herein.

As shown, the cell 155 in the second row (y) includes a first crown 153 (FIG. 1B) with the first set of apertures 150, 152 and a second crown (e.g., base 159 in FIG. 1B) with a second set of apertures 156, 158. The first crown 153 may be opposite the second crown (e.g., base 159). The first set of apertures 150, 152 may be configured to receive a first connecting element 168 (e.g., a bracket, support, a ring, a u-shaped member, etc.) to hold a first portion of the actuation member 166. The second set of apertures 156, 158 may be configured to receive a second connecting element 170 (e.g., a bracket, support, a ring, a u-shaped member, etc.) to hold a second portion of the actuation member 166. The first connecting element 168 and the second connecting element 170 may be coupled to frame 106 by passing from an outer diameter (exterior to the device 100) to an inner diameter (inner wall 172 of the device 100). For example, the first connecting element 168 may have a first end (not shown) and a second end (not shown) that forms the element. The first end may pass from the outer diameter through the first polymeric layer 174, through aperture 150, and through the second polymeric layer 178 into the inner diameter of device 100. Similarly, the second end may pass from the outer diameter, through the first polymeric layer 174, through the aperture 152, and through the second polymeric layer 178 into the inner diameter of device 100. In some embodiments, the first end and the second end may be coupled and reversibly sealed together. In some embodiments, the first end and the second end may be coupled and irreversibly sealed together. In some embodiments, the first end and the second end may be separately coupled to portions of frame 106 and/or bent to deter release from apertures 150, 152.

Similarly, the second connecting element 170 may have a first end (not shown) and a second end (not shown) that forms the element. The first end may pass from the outer diameter, through the first polymeric layer 174, through the aperture 156, and through the second polymeric layer 178 into the inner diameter of device 100. Similarly, the second end may pass from the outer diameter through the first polymeric layer 174, through the aperture 158, and through the second polymeric layer 178, and into the inner diameter of device 100. In some embodiments, the first end and the second end may be coupled and reversibly sealed together. In some embodiments, the first end and the second end may be coupled and irreversibly sealed together. In some embodiments, the first end and the second end may be separately coupled to portions of frame 106 and/or bent to deter release from apertures 156, 158.

In some embodiments, the actuation member 166 may be coupled to the frame 106, as described elsewhere herein, while being at least partially encased between the first polymeric covering (e.g., layer) 174 and the second polymeric covering (e.g., layer) 178. There may be a portion of the layers 174, 178 that are not laminated together along the path of the actuation member 166 to allow the actuation member 166 to move laterally and longitudinally when receiving instructions or movements for actuating the device 100. In such examples, the actuation member may be internal to the frame 106 of device 100 and at least partially encased between the first polymeric layer 174 and the second polymeric layer 178.

In some embodiments, the actuation member 166 is instead within a third layer (not shown) of polymeric material between an outer layer 178 and the third layer of polymeric material. In some embodiments, the actuation member 166 is instead within a fourth layer (not shown) of polymeric material within device 100 and between the inner layer 174 and the fourth layer of polymeric material.

For example, in FIG. 1C, the actuation member 166 is moveably coupled to the frame 106 by the connecting element 168. The connecting element 168 is coupled to the frame at aperture 150 and aperture 152 such that the actuation member 166 may be actuated longitudinally within a predefined range of movement. The actuation may be triggered by detected blood pressures within a blood vessel in which the device 100 is implanted. The actuation member 166 is also moveably coupled to the frame 106 by the connecting element 170. The connecting element 170 is coupled to the frame at aperture 156 and aperture 158 such that the actuation member 166 may be actuated longitudinally within a predefined range of movement. The actuation of actuation member 166 may be triggered by detected blood pressures within a blood vessel in which the device 100 is implanted. The actuation may be powered according to any of the power mechanisms described herein. The actuation may cause the outflow crowns 122a-122i to collapse inward toward central axis (C) (e.g., FIG. 1F) or expand outward away from central axis (C) (e.g., FIG. 1C).

The connecting elements 168, 170 may be coupled to respective apertures 150, 152, 156, 158 by welding, soldering, threading, braiding, or otherwise attaching therethrough. In some examples, each aperture 150, 152 may be arranged to receive an end portion of a bracket or shaped wire (e.g., support 168 in FIG. 1C). The end portion may be welded, soldered, threaded, braided, or otherwise attached through the apertures 150, 152. Similarly, each aperture 156, 158 may be arranged to receive an end portion of a bracket (e.g., support 170 in FIG. 1C). The end portion may be welded, soldered, threaded, braided, or otherwise attached through the apertures 156, 158. In some examples, the supports 168, 170 may be arcuate, partial circles, partial ovals, or the like.

In some examples, the connecting elements may be formed of stainless steel or other material capable of being coupled, attached, or otherwise connected to the frame 106. Similarly, the actuation member 166 may be formed of stainless steel. The actuation member 166 may be tube-shaped and may be coupled to the frame 106 using one or more connecting elements 168, 170, etc. In some examples, the actuation member 166 may receive a cinching wire therethrough as described elsewhere herein.

FIG. 1D is a perspective view of the frame 106 of device 100 depicting example polymeric coverings. For example, the device 100 includes an inner wall 172 (e.g., inner surface) with a first polymeric covering 174 and an outer wall 176 (e.g., outer surface) with a second polymeric covering 178. The first and second polymeric coverings 174, 178 may be composed of a material capable of heat shrinking and/or lamination such that portions of each covering are coupled at locations along the stent walls 172, 176. For example, the first polymeric covering 174 and the second polymeric covering 178 may be formed of thermoplastic polyurethane or polyolefin or other polymer described herein. The first polymeric covering 174 may be coupled to portions of the second polymeric covering 178 to form a circumferentially sealed space 180 that is defined to extend around a portion of a circumference of the outer wall 176 of the frame. In some examples, the circumferentially sealed space 180 may also extend longitudinally along at least a portion of the frame in longitudinal axis (L) and around the circumference of the outer wall. In some examples, the circumferentially sealed space 180 extends around an entire circumference of the outer wall of the frame 106 and specifically to extend above (e.g., adjacent to and above a joint) and below (e.g., adjacent to and below the joint) the plurality of joints 160 (FIG. 1B). In some examples, coupling the first polymeric covering 174 to the portions of the second polymeric covering 178 may include laminating the portions of the second polymeric covering 178 to the first polymeric covering 174. In some examples, the first polymeric covering 174 is optional. In some examples, the second polymeric covering 178 is optional. For example, the first (inner) polymeric covering 174 may be coupled to portions of the skirt membrane 186 rather than portions of the second polymeric covering 178.

In some examples, the frame 106 is formed from a plurality of struts (e.g., cells, stent rings, diamond-shaped structures, or the like) spaced from the inflow end 102 to the outflow end 104 and extending from each other around the frame to form two or more rows of cells (e.g., row (x), row (y), and/or row (z) in FIG. 1A). In such an example, the first polymeric covering 174 and the second polymeric covering 178 may be heat shrunk together around the frame 106 at the inflow end 102 to form a substantially circular opening at the inflow end 102. In some examples, the polymeric coverings 174, 178 may provide an advantage of reducing or eliminating clotting and/or blood stasis within cells and struts of the device 100 by providing an effective seal around the inflow end 102 and the outflow end 104 to prohibit blood from flowing across the struts of the frame 106.

In addition, a number of crowns 126a, 126b, 126c, 126d, 126e, 126f, 126g, 126h, and 126i in the frame 106 at the inflow end 102 may be mitered around to form cut reliefs (e.g., cut relief 181a, cut relief 181b, etc., of FIG. 1D) between each crown at the inflow end 102. The cut reliefs may provide an advantage of reducing force generated at implantation by reducing material that may cause drag or pressure on the vessel. The cut reliefs (e.g., cut relief 181a, cut relief 181b, etc.) may include open space formed by cutting into the first polymeric covering 174 and the second polymeric covering 178 from about 0.1 millimeter to about 15 millimeters from an edge formed by the coupled first polymeric covering 174 and the second polymeric covering 178. In some examples, the cut reliefs (e.g., cut relief 181a, cut relief 181b, etc.) are shaped based on a shape of an end portion of the inflow end 102 of the frame 106. While the example cut reliefs shown in FIGS. 1D, 1E, 1F, 1G, 1H, and 1K are substantially v-shaped, any shape that generally follows along at least a portion of the frame 106 at end 102 may be used, including, but not limited to u-shaped, deep v-shaped, square shaped, partial circle or oval-shaped, etc.

In some examples, the first and second polymeric coverings 174, 178 extend beyond the outflow end 104. When the coverings 174, 178 are laminated (e.g., heat shrunk, adhered together, laminated, etc.), device 100 may be arranged in a substantially tubular-shaped device with a substantially annular cross section at the inflow end 102 and a substantially flower-shaped, star-shaped, or polygon-shaped cross section at the outflow end 104. For example, the polymeric coverings 174, 178 may be shaped to form indentations between each crown at the outflow end 104 to form a cross section of the device 100 that is at least partially cinched from a circular cross section at the inflow end 102 to a star or flower-shaped cross section at the outflow end 104 when the implantable device is configured in an unrestricted blood flow state. Further narrowing of the device 100 at the outflow end 104 may be performed using actuation member 166 to adjust the device 100 to further cinch the outflow end resulting in reversibly reducing or closing the substantially star-shaped or polygon-shaped cross section at the outflow end 104, when the implantable device is configured in a restricted or partially restricted blood flow state, for example.

FIG. 1E is a side view of the frame 106 of device 100 including a plurality of eyelets (e.g., eyelets 182a, 182b, 182c, etc.) for receiving an actuatable control wire 183. In some examples, the eyelets 182a, 182b, 182c, etc. are each formed of a shape memory alloy (e.g., Nitinol® or a combination of Nitinol® and palladium, gold, or their alloys or one or more polymers). The eyelets may be coupled to the frame at a predefined distance from the outflow end 104 and around the circumference of the frame 106. The predefined distance may be about 10 to about 20 mm from the outflow end 104; about 10 mm to about 12 mm from the outflow end 104; about 12 mm to about 14 mm from the outflow end 104; about 14 mm to about 16 mm from the outflow end 104; about 16 mm to about 18 mm from the outflow end 104; about 18 mm to about 20 mm from the outflow end 104.

Each eyelet 182a, etc. may include a bracket with a first end (e.g., end 184a) and a second end (e.g., 184b) inline with the first end 184a. Each first end may be coupled to a first aperture (e.g., aperture 162 of FIG. 1B) defined on the frame 106 and each second end is coupled to a second aperture (e.g., aperture 164 of FIG. 1B) defined on the frame 106. The first aperture 162 and the second aperture 164 may be along the longitudinal axis (L) of the frame 106.

The eyelets 182a, etc. may be coupled to the frame 106 at locations around the outer circumference of the frame 106. The eyelets 182a, etc. may be arranged substantially perpendicular to the longitudinal axis (L) and within a threshold distance of the distal/outflow end 104 of the frame 106. In general, the eyelets 182a, etc. may each have a through hole arranged to receive the actuatable control wire 183. For example, the actuatable control wire 183 may be threaded through the through hole of the eyelets 182a, 182b, 182c, etc. and into actuation member 166, as shown by wire portion 183a and wire portion 183b. eyelets 182a, etc., may be a plurality of brackets coupled to the frame 106 such that when the control wire 183 is threaded through the through holes of the plurality of brackets, the device 100 may be ready to be actuated. For example, the device 100 may be actuated by pulling or releasing the control wire 183. Pulling or releasing the control wire can be performed by moving the actuation member 166 away from the outflow end 104 or toward the outflow end 104, respectively. When pulling the actuation member 166 away from the outflow end 104, the eyelets 182a, etc. move radially (e.g., cinching each crown 122a-122i together) toward the central axis (C) of the frame 106. For example, actuating the control wire 183 reversibly cinches the outflow end 104 toward the central axis (C) by bringing the crowns 122a--122i together (or closer to another crown in the crowns 122a-122i) to occlude or partially occlude a blood vessel in which the device 100 is implanted.

The eyelets 182a, etc., when threaded with control wire 183, may form a waist member 185 formed by a plurality of joints 160 (FIG. 1B) within the circumferentially sealed space 180 (FIG. 1D). The waist member 185 may be disposed around the outer wall 176 of the frame 106 and beneath the inner polymeric covering 174. The circumferentially sealed space 180 may be defined to extend around the waist member 185 to allow the eyelets 182a, etc. to be cinched and uncinched within the space 180 while avoiding contact with blood from the vessel that may be flowing through the device 100. Although one waist member 185 is depicted on device 100, any number of waist members 185 may be utilized to cinch and uncinch portions of device 100 to modulate blood flow in a vessel in which device 100 is implanted.

In operation, the actuation member 166 (coupled to the frame 106 between the first polymeric covering 174 and the second polymeric covering 178) may actuate the waist member 185 to flexibly collapse the outflow end 104 of the frame 106 toward a central axis (C) of the frame 106, or expand the outflow end 104 away from the central axis (C), by actuating of the control wire 183 coupled to the waist member 185 and the actuation member 166. For example, the control wire 183 may be flexible and may function as a lasso to be actuated by an actuation member 166 to radially expand and constrict (uniformly or nonuniformly) a perimeter of the end portion of the outflow end 104 to function as an adjustable blood flow restrictor.

In some examples, the device 100 further includes a skirt membrane 186. The skirt membrane 186 may be wrapped around an exterior surface of the second polymeric covering 178 and disposed offset from a lateral centerline (e.g., bisect B in FIG. 1A) at a midpoint of the frame 106 and toward the inflow end 102 of the frame 106. An inflow perimeter 187 of the skirt membrane 186 may end within a threshold distance of the inflow end 102 of the frame 106. For example, the threshold distance may be about 8 mm to about 12 mm from the inflow perimeter 187; about 8 mm to about 9 mm from the inflow perimeter 187; about 9 mm to about 10 mm from the inflow perimeter 187; about 10 mm to about 11 mm from the inflow perimeter 187; or about 11 mm to about 12 mm from the inflow perimeter 187. A length l ₛ₁ of the skirt membrane 186 may be about 15 mm to about 19 mm; about 15 to about 16 mm; about 16 mm to about 17 mm; about 17 mm to about 18 mm; or about 18 mm to about 19 mm.

The skirt membrane 186 may function to reduce blood stasis and/or pooling around the implantable device 100. For example, the skirt membrane 186 may be at least partially incorporated into an inner wall of the blood vessel to reduce or eliminate clotting and/or blood stasis within cells and struts of the device 100 by providing an effective seal between the skirt membrane 186 and the blood vessel wall.

In a non-limiting example, the skirt membrane 186 may be incorporated into an inner wall of the vena cava and may be positioned to seal an entrance to at least one additional blood vessel (or junction) branching from the vena cava when the implantable device 100 is implanted in the vena cava. For example, the skirt membrane 186 may be positioned in the IVC or the SVC and aligned to seal or block an entrance to the azygos junction. Sealing, blocking, or occluding the azygos junction can ensure that blood does not bypass the device 100, but instead flows through an inner diameter of device 100.

In some examples, the skirt membrane 186 may be formed of poly-delta-valerolactone (PVL). In some examples, the skirt membrane 186 may be formed of PVL and another polymer. In some examples, the skirt membrane 186 may be formed of mesh or braided metal that may be coated. In some examples, an inner surface of the skirt membrane 186 may be coupled to the outer polymeric layer 178. In some examples, the skirt membrane 186 may be sewn, sutured, or otherwise affixed to a portion of the second polymeric covering 178.

In this example, the device 100 is shown in an unrestricted blood flow state. The unrestricted blood flow state may represent a state of device 100 in which both an inflow end 102 and an outflow end 104 are open to receive fluid (e.g., blood, drugs, saline, etc.). The fluid flows through the inflow end 102 and through a lumen of the device 100 and out of the outflow end 104. For example, the device 100 may be in the expanded state when both the inflow end 102 and the outflow end 104 are open to receive fluid (e.g., blood, drugs, saline, etc.) therethrough when the device 100 is implanted in a blood vessel.

FIG. 1F is a side view of the frame 106 of FIG. 1E in an at least partially collapsed position. For example, the crowns 122a-122i at the outflow end are shown at least partially collapsed to at least partially block blood flow through a lumen of the device 100 when the device 100 is implanted in a vessel.

Positioning the crowns 122a-122i in a particular arrangement may be performed using the actuation member 166. For example, actuating the actuation member 166 may place the device into a restricted blood flow state, a partially restricted blood flow state, or an unrestricted blood flow state by positioning one or more crowns 122a-122i to adjust the outflow end 104. The crowns 122a-122i may be moved to any of a plurality of positions between expanded and collapsed. Such positions may include at least an expanded position to allow the blood flow through the blood vessel, a partially expanded position to partially occlude the blood vessel, and a collapsed position to block the outflow end 104 to occlude the blood vessel.

As used herein, examples referring to devices that allow blood to flow through the blood vessel may represent the blocking or unblocking of blood flow through a lumen of the particular device (rather than through the vessel itself) when the device is implanted. For example, portions of the blood vessel may be implanted with one or more of the devices described herein and those portions may be allowed or disallowed to flow blood through the lumen of the device within the vessel resulting in partially blocking or unblocking flow through the portions of the blood vessel implanted with such a device.

In some examples, the frame 106 may be substantially tubular-shaped with a substantially annular cross section at the inflow end 102 and a substantially star-shaped or polygon-shaped cross section at the outflow end 104 when the implantable device 100 is in an unrestricted blood flow state. The frame 106 may be adjustable to form a cinched portion at the outflow end 104 where the cinching may result in reversibly reducing or closing the substantially star-shaped or polygon-shaped cross section at the outflow end 104 when the implantable device is in a restricted or partially restricted blood flow state.

In this example, the device 100 is shown in a restricted (or partially restricted) blood flow state. The restricted/partially restricted blood flow state may represent a state of device 100 in which both the inflow end 102 is open to receive fluid, but the outflow end 104 is partially or fully collapsed to block or partially slow fluid through the outflow end 104. For example, the device 100 may be in a collapsed or partially collapsed state when the outflow end 104 is at least partially blocking or slowing fluid therethrough when the device 100 is implanted in a blood vessel.

In some examples, the control wire 183 may be actuatable and coupled to a power source (e.g., power source 414) electrically coupled to device 100. The power source may trigger actuation (or be caused to trigger actuation) of the control wire 183. Actuating the control wire 183 may result in positioning the outflow end 104 and/or device 100, in general, in an unrestricted blood flow state or a restricted blood flow state. To actuate the control wire 183, the actuation member 166 may use the power source associated with or coupled to device 100 to induce changes in blood flow states of at least a portion of device 100. In some examples, the actuation member 166 may use passively induced movement. For example, passively moving a portion of the device 100 may include manually actuating pull wires (e.g., sutures, actuation wires/cords/elements, etc.) and/or anatomy responses (e.g., changes in vessel inner diameter, intra-vessel pressure, pressure changes in a thoracic cavity versus an abdominal cavity, intercostal region movement as a result of respiration, etc.).

In some examples, the actuation member 166 for actuating the control wire 183 of the device 100 may include an actuator (as described herein elsewhere) that may be coupled to the control wire 183 of the device 100, coupled to a first magnet to induce rotation of the actuator, and coupled to a control device communicatively coupled to the actuator. In some examples, the first magnet is a permanent magnet, and the second magnet is a permanent magnet. In some examples, the first magnet is a permanent magnet, and the second magnet is an electromagnet. In some examples, the actuation member 166 may be a magnetically driven actuator. In such an example, the control device may include a second magnet for generating a changing magnetic field pole direction to cause rotation of the first magnet and operation of the control wire 183 and device movement to the unrestricted blood flow state or to the restricted blood flow state. For example, the actuation member 166 may cause rotation of the second magnet in a first direction to induce rotation of the first magnet, thereby causing the actuator to tension the control wire 183 to cause the outflow end 104 to radially collapse inward and toward the central axis (C). For example, the first direction of rotation of the second magnet may attract the first magnet.

The actuation member 166 may also cause rotation of the second magnet in a second direction to induce rotation of the first magnet, thereby causing the actuator to release tension in the control wire 183 to cause the outflow end 104 to radially open crowns 122a-122i. For example, the second direction of rotation of the second magnet may repel the first magnet.

In some examples, the control device is implanted in the same user (e.g., subject) that the device 100 is implanted within. The control device may be implanted adjacent to the device 100 or remote from the device 100. In some examples, the control device is implanted subcutaneously in the subject. In some examples, the control device is disposed external to a body of a subject (e.g., a user) associated with the device.

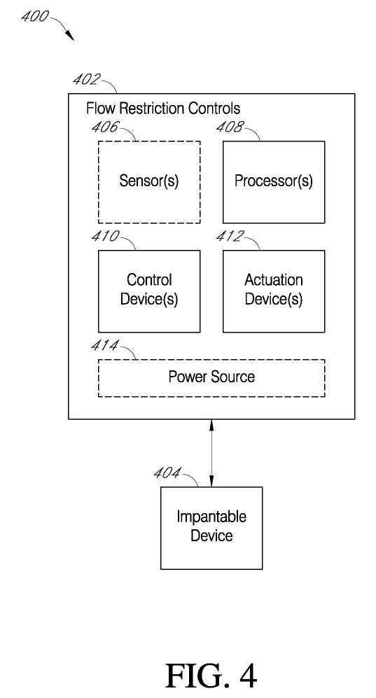

In some examples, the device 100 may further include a sensor (e.g., sensor 406 of FIG. 4) for detecting a pressure in the blood vessel, a microprocessor (e.g., processor 408) electrically coupled to the sensor 406, and/or a power source (e.g., power source 414) electrically coupled to an actuator (e.g., actuation device 412, actuation member 166, actuation device 508) associated with device 100, the microprocessor 408, and the sensor 406. For example, the sensor 406 may sense characteristics of blood flow in the blood vessel (e.g., blood pressure) and may cause the microprocessor 408 to provide signals to the actuator (e.g., actuation device 412) and/or actuation member 166 and/or control wire 183 (e.g., control devices 410). In operation, the microprocessor 408 can receive a signal from the sensor 406 that is indicative of a pressure in the blood vessel. The microprocessor 408 can process the signal and generate and provide a control signal (e.g., via control devices 410) to tension the control devices 410, or release tension in the control devices 410 based on the sensed pressure in the blood vessel.

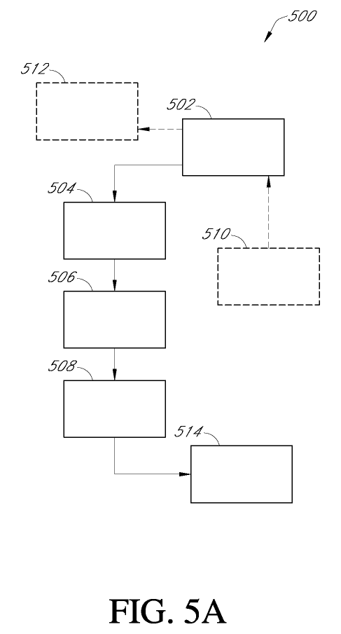

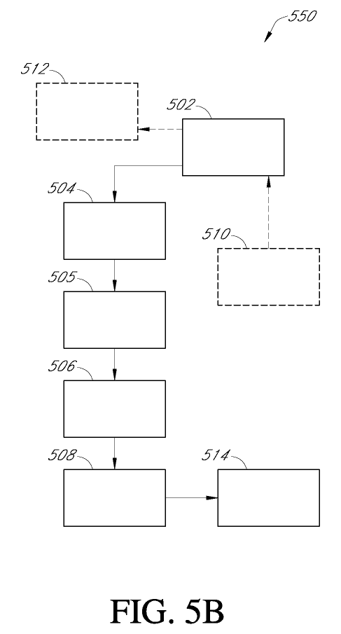

In some examples, the sensor 406 may be communicatively coupled to device 100. The sensor 406 may include one or more of an image sensor, a strain gauge, a piezoelectric sensor, a capacitance sensor, and/or a vacuum pressure sensor. If the device 100 is coupled to a power source (e.g., power source 414), the power source may include an induction coil. The induction coil may be used to operate one or more of such magnets, as described in further detail in FIGS. 5A-5B.

In operation, the device 100 may receive a signal from an actuator that triggers the actuation member 166 to cause actuation of the control wire 183 and in turn causes a radial collapse of the frame 106 at the outflow end 104. Such an actuation of the control wire 183 may cause the control wire 183 to be tensioned and to pull the crowns 122a-122i radially toward the central axis (C) to collapse or partially collapse the outflow end 104. In addition, the outflow end 104 may be triggered to radially expand away from the central axis (C) of the expandable frame 106, in response to an actuation of the control wire 183 responsive to actuation of the actuation member 166 connected to the control wire 183 to cause the control wire 183 to release the tension and to release tension upon the eyelets 182a, etc., and to release crowns 122a-122i radially away from the central axis (C) to expand or partially expand the outflow end 104 of the frame 106.

The device 100 may include a power source (e.g., power source 414) coupled to the control wire 183. The power source may include a battery or a wall outlet that may be electrically connected to the control wire 183 or another portion of device 100. The electrical connection may allow active powering of device 100 operations. In such an example, a processor may be utilized to send and/or receive signals to activate device operations via the actuation device 412, such as the actuation member 166 and/or control wire 183. In some examples, the actuation device can send a first signal to the control wire 183 to activate application of tension to the control wire 183. For example, a processor (e.g., processor 408) may be programmed to trigger tensioning of the control wire 183 in response to detecting a particular condition of the blood vessel or the device 100. The tensioning of the control wire 183 may result in cinching the outflow end 104 to place the device 100 in a restrictive blood flow state. Similarly, the actuation device 412 can send a second signal to the control wire 183 to activate releasing of the tension from the control wire 183 in response to detecting another condition of the blood vessel or the device 100. For example, a processor (e.g., processor 408) may be programmed to trigger a release of tension in the wire 183 in response to detecting a particular condition of the blood vessel or the device 100. The release of the tension of the control wire 183 may result in uncinching the outflow end 104 of the frame 106 to place the device in an unrestrictive blood flow state.

FIG. 1G is a side view of the frame 106 of FIG. 1E and a zoomed in view 189 of a waist member 185 installed therein. For example, the waist member 185 is arranged on the frame 106 and between the first polymeric layer 174 and the second polymeric layer 178 and within the circumferentially sealed space 180 defined to extend around a portion of a circumference of the outer wall 176 of the frame 106. The waist member 185 includes at least a plurality of eyelets 182a, 182b, 182c, etc., which may be coupled or otherwise attached to the frame 106 at locations around the circumference c and arranged substantially perpendicular to the longitudinal axis (L) and within a threshold distance d of the outflow end 104 of the frame 106. The eyelets 182a, 182b, 182c, etc. may be threaded with the control wire 183 to allow cinching and uncinching of waist member 185 to cause crowns 122a-122i to move radially inward or radially outward.

The threshold distance d may be about 10 mm to about 16 mm from the outflow end 104; about 10 mm to about 11 mm from the outflow end 104; about 11 mm to about 12 mm from the outflow end 104; about 12 mm to about 13 mm from the outflow end 104; about 13 mm to about 14 mm from the outflow end 104; about 14 mm to about 15 mm from the outflow end 104; about 15 mm to about 16mm from the outflow end 104.

FIG. 1H is a side view of a frame (e.g., frame 106) depicting an example configuration of supports and detents. For example, the frame 106 shown in FIG. 1H includes the support 168 and support 170 along the actuation member 166. The supports 168, 170 may be formed on and/or otherwise coupled to the frame 106. The supports 168, 170 may be coplanar with the longitudinal axis (L) and radially bent to allow the member 166 to pass through each support 168, 170.

The frame 106 may further include a first detent 190 and a second detent 191. The first detent 190 may function to inhibit movement of the actuation member 166 beyond a first threshold distance (dₜᵣ₁) along a first direction of arrow 192, for example. The first direction may be toward the inflow end 102 of the frame 106. The frame 106 may further include a second detent 191 to inhibit movement of the actuation member 166 beyond a second threshold distance (dₜᵣ₂) along a second direction of arrow 192, for example. The second direction may be toward the outflow end 104 of the frame 106.

The first and second threshold distances [(dₜᵣ₁) , (dₜᵣ₂)] in which detents 190, 191 can prevent axial movement of the actuation member 166 may be about 0.1 mm to about 0.7 mm; about 0.1 mm to about 0.2 mm; about 0.2 mm to about 0.3 mm; about 0.3 mm to about 0.4 mm; about 0.4 mm to about 0.5 mm; about 0.5 mm to about 0.6 mm; or about 0.6 mm to about 0.7 mm.

The first and second detents 190, 191 may be stoppers, bungs, or other barricade for stopping movement of the actuation member 166 beyond a boundary of the respective detent 190, 191. The detents 190, 191 may be coupled, welded, adhered, or otherwise attached to a portion of the frame 106. The detents 190, 191 may be formed of any material suitable for implantation in a blood vessel to provide a blocking of movement beyond a particular boundary of the respective detent 190, 191. In some examples, the detents 190, 191 may be made of nylon, synthetic polymer materials (e.g., silicone, polydioxanone, polyglycolic acid, polyglyconate, polylactic acid, etc.), metal (e.g., Nitinol®, palladium, stainless steel, gold and their alloys, etc.), or a combination thereof.

FIG. 1I is a block diagram depicting an example configuration of supports and detents. In this example, support 168 is shown at a frame junction 193a and support 170 is shown at a frame junction 193b. Each support 168, 170 may extend in the same direction from the respective junction 193a, 193b prior to bending of the supports during manufacturing, for example. The detent 190 may block the support 168 from moving beyond the detent 190 (i.e., further along junction 193a toward the inflow end 102). The detent 191 may block the support 170 from moving beyond the detent 191 (i.e., further along junction 193b toward the outflow end 104).

FIG. 1J is another example configuration of supports and detents for the frame of FIG. 1E. In this example, the support 168 is positioned to extend in a direction along the junction 193a that is opposite the direction that support 170 extends along the junction 193c. The arrangement of supports 168, 170 may represent support placement prior to bending of the supports during manufacturing, for example.

FIG. 1K is a side view of a frame 106 depicting an example configuration of supports for moving and inhibiting movement of actuation member 166. In this example, the frame 106 includes a first support (e.g., support 168) along a first portion of the actuation member 166 and a second support (e.g., support 170) along a second portion of the actuation member 166. The first support 168 and the second support 170 may include rings, brackets, wires, mesh, or other shape for supporting and/or holding the actuation member 166 along the frame 106. In some examples, the first support 168 and the second support 170 may be formed of metal, polymers, copolymers, textiles, tissues, or a combination thereof.

FIG. 1L is yet an example configuration of supports 168, 170 of FIG. 1K. The first support 168 may be aligned on the frame 106 along a frame junction 193a. The second support 170 may be aligned on the frame 106 along a frame junction 193b. To stop axial movement, the first support 168 may be contained within a first groove 194a of the actuation member 166 while the second support 170 may be contained within a second groove 194b of the actuation member 166. The first groove 194a and the second groove 194b may be parallel to one another along the longitudinal axis (L). In this example, each support 168, 170 may extend in the same direction from the respective junctions 193a, 193b prior to bending of the supports during manufacturing, for example.

FIG. 1M is another example configuration of supports. In this example, the support 168 is positioned to extend in a direction along the junction 193a that is opposite the direction that support 170 extends along the junction 193c. To stop axial movement, the first support 168 may be contained within a first groove 194a of the actuation member 166 while the second support 170 may be contained within a second groove 194b of the actuation member 166. The first groove 194a and the second groove 194b may be parallel to one another along the longitudinal axis (L). In this example, each support 168, 170 may extend in the same direction from the respective junctions 193a, 193b prior to bending of the supports during manufacturing, for example. In some examples, the grooves 194a, 194b may function to limit a range of movement of the actuation member 166 by receiving and detaining supports 168, 170 within the respective grooves 194a, 194b.

While the supports 168, 170 are depicted at a right angle in the figures described herein, other angles of orientation are possible. For example, the supports 168, 170 may be angled from the surface of the actuation member 166 at about 25 degrees to about 60 degrees; 25 degrees to about 30 degrees; about 30 degrees to about 35 degrees; about 35 degrees to about 40 degrees; about 40 degrees to about 45 degrees; about 45 degrees to about 50 degrees; about 50 degrees to about 55 degrees; or about 55 degrees to about 60 degrees.

In some examples, the frame 106 has a pro-endothelialization coating, such that the frame 106, frame covering (e.g., polymeric coverings 174, 178), and/or other frame layers (e.g., skirt membrane 186) can be at least partially incorporated into an inner wall of the blood vessel. This incorporation may allow a site of the device 100 to maintain a non-thrombogenic, non-immunogenic environment with respect to the device 100. For example, the coating of the frame 106 may be any pro-endothelial factor including, but not limited to, endothelial growth factor, vascular endothelial growth factor, or any related compound.

In some examples, the device 100 may be an implantable device for dynamically occluding a blood vessel such as a vein or the vena cava (e.g., an SVC or IVC). For example, the device 100 may be sized for implantation in a vein. In some examples, the device 100 may be sized for implantation in the vena cava. For example, the device 100 may include a frame 106 having a lumen extended therethrough and sized for implantation into particular vasculature. In a non-limiting example, the frame 106 may have a diameter of about 3 mm to about 6 mm; about 3 mm to about 3.2 mm; about 3.2 mm to about 3.6 mm; about 3.6 mm to about 3.9 mm; about 3.9 mm to about 4.2 mm; about 4.2 mm to about 4.5 mm; about 4.5 mm to about 4.8 mm; about 4.8 mm to about 5.1 mm; about 5.1 mm to about 5.3 mm; about 5.3 mm to about 5.6 mm; or about 5.6 mm to about 6 mm.

In some examples, the frame 106 may be a tubular frame with an outflow end 104 that may adjust to any number of positions between expanded and collapsed. The positions may include at least an expanded position to allow the blood flow through the blood vessel, a partially expanded position configured to partially occlude the blood vessel, and a collapsed position configured to block the outflow end to occlude the blood vessel.

The frame 106 may be a stent (or braid) constructed of metal wire (e.g., stainless steel, platinum, palladium, gold and their alloys, Nitinol® wire or another shape memory alloy) or some combination thereof, or other material suitable for implantation in the human body. In some examples, the frame 106 is a bare metal stent, such that the frame 106 may be arranged to be at least partially incorporated into an inner wall of the blood vessel. In some examples, the frame 106 includes one or more additional coverings such as sleeves, skirts, etc., as described elsewhere herein.

The implantable device 100 may further include a sealing element (e.g., polymeric coverings 174, 178 described elsewhere herein) that may be disposed along an exterior of the frame (or surface or portion thereof) and along an interior of the frame (or surface or portion thereof). The exterior may be opposite the interior of the frame. The exterior may represent any number of portions or surfaces along and around the exterior of the frame 106. The interior may represent any number of portions or surfaces along and around the interior of the frame 106 (and around the lumen). The sealing element may function to seal portions of the frame 106 between the exterior and the interior of the frame 106 to ensure that blood and tissue remains outside of the frame componentry.

In general, the frame 106 may include the inflow end 102 and an outflow end 104 with an inner wall 172 and an outer wall 176. The sealing element may include one or more portions to cover all or a portion of the inner wall 172. The sealing element may include one or more portions to cover all or a portion of the outer wall 176. In some examples, the sealing element may include a first polymeric covering 174 and a second polymeric covering 178. The one or more portions of the sealing element may be sealed together to form a seal or one or more sealed spaces surrounding all or a part of the frame 106. For example, the first polymeric covering 174 may be coupled to portions of the second polymeric covering 178 to form a circumferentially sealed space defined to extend around a portion of a circumference of the outer wall of the frame 106. In such examples, portions of the first polymeric covering 174 and the second polymeric covering 178 may be heat shrunk together around portions of the frame 106. In some examples, the sealing element is substantially composed of polymer and may include a first covering disposed along an exterior of the frame and a second covering disposed along an interior of the frame.

In some embodiments, the actuation member 166 may be coupled to the frame 106, as described elsewhere herein, while being at least partially encased between the first polymeric covering 174 and the second polymeric covering 178. There may be a portion of the layers 174, 178 that are not laminated together along the path of the actuation member 166 to allow the actuation member 166 to move laterally and longitudinally when receiving instructions or movements for actuating the device 100. In such examples, the actuation member may be internal to the frame 106 of device 100 and at least partially encased between the first polymeric layer 174 and the second polymeric layer 178.

In some embodiments, the actuation member 166 is instead within a third layer (not shown) of polymeric material between an outer layer 178 and the third layer of polymeric material. In some embodiments, the actuation member 166 is instead within a fourth layer (not shown) of polymeric material within device 100 and between the inner layer 174 and the fourth layer of polymeric material.

In some examples, the implantable device 100 may further include an actuation member (e.g., actuation member 166) coupled to the frame 106. The actuation member 166 may alter a diameter of the frame 106 to modulate blood flow through the lumen (e.g., the inner cavity of the frame 106 from the inflow end 102 to the outflow end 104. In some examples, the actuation member 166 may be at least partially sealed within the sealing element and/or portions of the sealing element.

In some examples, the actuation member 166 may actuate a portion of the outflow end 104 of the frame 106 to alter the diameter of the frame 106. For example, the actuation member may alter the diameter of the frame responsive to receiving a signal from a pressure sensor (e.g., sensor 406 of FIG. 4) coupled to the frame 106. In some examples, the actuation member 166 may be used to manually trigger all or a portion of the outflow end to collapse inward toward the central axis (C). In some examples, an actuator may tension a control wire (e.g., control wire 183) to cause the outflow end 104 to radially collapse inward and toward the central axis (C). In examples in which the actuation member/device, control wire, or waist member, as described elsewhere herein, extends around a periphery of the frame 106, the actuation may trigger a symmetrical or asymmetrical reduction of the diameter over a portion of the lumen.

In some examples, the actuation member 166 may be controlled by a power source (e.g., power source 414 of FIG. 4), and may also be coupled to the control wire 183 to cause actuation of the actuation member 166. For example, the actuation member 166 may send a first signal (using a power source, processor, and/or sensor) to the control wire 183 to activate application of tension to the control wire 183. Similarly, the actuation member 166 may send a second signal (using a power source, processor, and/or sensor) to the control wire 183 to activate release of the tension from the control wire 183. In some examples, the signal may be mechanically received responsive to a tension or pull on the actuation member 166.

In some examples, the sensors (e.g., sensor 406 of FIG. 4) described herein may be coupled to a processor adapted to improve heart function by causing actuation of the actuation member 166 responsive to receiving the signal from the pressure sensor(s), which may cause opening, partially opening, partially closing, or fully closing of a portion of the lumen.

In examples of device 100 that include a waist member, such waist members may be disposed along a portion of the frame 106 and within the sealing element. The waist member may function to flexibly collapse the outflow end 104 of the frame 106 or expand the outflow end 104, in response to actuation of the control wire 183, for example, when coupled to the waist member and the actuation member. In such examples, the actuation member may be controlled by a power source and coupled to the control wire.

In operation, the device 100 may receive a signal from an actuator that triggers the actuation member 166 to cause actuation of the control wire 183 and in turn causes a radial collapse of the frame 106 at the outflow end 104. Such an actuation of the control wire 183 may cause the control wire 183 to be tensioned and to constrict a portion of the frame 106 radially inward toward the central axis (C) to collapse or partially collapse the portion of the frame 106. In addition, the outflow end 104 may be triggered to radially expand away from the central axis (C) of the expandable frame 106, in response to an actuation of the control wire 183 responsive to actuation of the actuation member 166 connected to the control wire 183 to cause the control wire 183 to release the tension and to release tension in the portion of the frame 106 to expand or partially expand the previously constricted portion of the frame 106.

The power source may include a battery or a wall outlet that may be electrically connected to the control wire 183 or another portion of device 100. The electrical connection may allow active powering of device 100 operations. In such an example, a processor may be utilized to send and/or receive signals to activate device operations on the actuation device 412, such as the actuation member 166 and/or control wire 183.

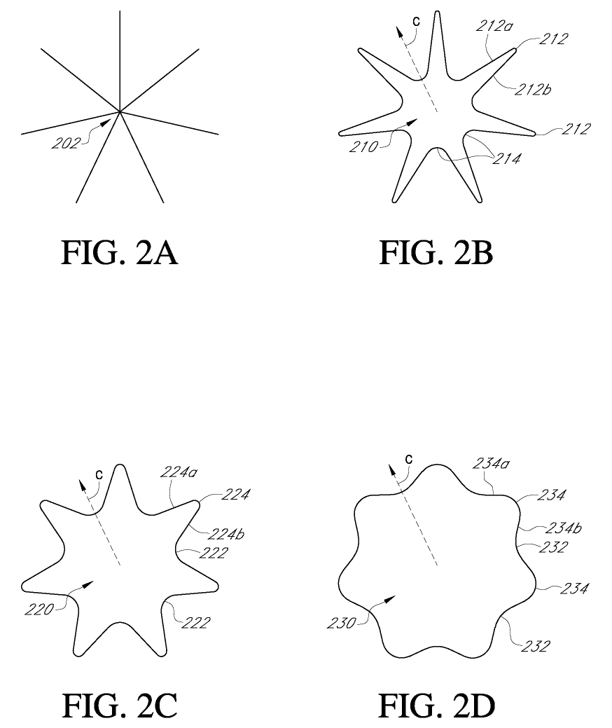

FIG. 2A is an example cross-sectional view of an outflow end of the devices described herein in a restricted blood flow state. For example, the outflow end 104 of device 100 may resemble a substantially closed outflow cross section 202 when the device 100 is in the restricted state. As shown, the crowns 122a-122i of the frame 106 may be radially closed to form cross section 202 and fully block or at least partially block blood flow through a lumen of the device when the device is implanted.

FIG. 2B is an example cross-sectional view of an outflow end of the devices described herein in a partially restricted blood flow state. For example, the outflow end 104 of device 100 may resemble a partially closed outflow cross section 210 when the device 100 is in the partially restricted state. The cross section 210 is depicted as a central cavity formed by an inner perimeter/surface with a plurality of bends with protrusions (e.g., protrusions 212) and indentations (e.g., indentations 214) formed by sidewall 212a and sidewall 212b, for example. The apex of each indentation 214 may represent one of the crowns 122a-122i. Although seven apexes are depicted, any number of apexes may be represented based on a number of crowns that are at the outflow end 104 of frame 106.

In operation, actuation of the control wire 183 may cause the control wire 183 to be tensioned and to pull the crowns 122a-122i (e.g., indentations 214) radially or semi-radially (e.g., asymmetrically toward or away from the central axis (C)) to collapse or partially collapse the cross section 210. In addition, the outflow end 104 may be triggered to radially expand away from the central axis (C) of the expandable frame 106, in response to an actuation of the control wire 183 responsive to actuation of the actuation member 166 connected to the control wire 183 to cause the control wire 183 to release the tension on the eyelets 182a, etc., and to release crowns 122a-122i (e.g., indentations 214) radially or semi-radially away from the central axis (C) to expand or partially expand the cross section 210.

FIG. 2C is another example cross-sectional view of an outflow end of the devices described herein in a partially restricted blood flow state. In this example, a cross section 220 may be formed by indentations 222 and protrusions 224 which are formed by angling sidewall 224a and sidewall 224b to couple together at one end, for example. The indentations are arranged further from the central axis (C) than the locations of indentations 214 causing additional area through the cross section 220 to receive additional blood flow. Protrusions 224 may have sidewalls 224a and 224b that are angled at a shallower angle than the angled arrangement of sidewalls 212a, 212b associated with protrusions 212, for example.

FIG. 2D is an example cross-sectional view of an outflow end of the devices described herein in an unrestricted blood flow state. In this example, a cross section 230 may be formed by indentations 232 and protrusions 234 which are formed by coupling together sidewall 234a and sidewall 234b at one end, for example. The indentations are arranged further from the central axis (C) than the locations of indentations 222 and indentations 214 causing additional area through the cross section 220 to receive additional blood flow.

Although a flower-shaped or serpentine shaped cross section is depicted in FIGS. 2A-2D, any cross section configuration or conformation is contemplated herein including a substantially circular cross section, a beveled cross section, a star-shaped cross section, or the like.

FIG. 3 is an example perspective view of a flow restricting device 100 and an example delivery system 300 for deploying the flow restricting device 100 in a blood vessel. The delivery system 300 may also be used to recapture device 100 to retrieve or move the device 100 from prior implantation.

The delivery system 300 may include an outer sheath 302 for reversibly receiving a pusher 304. The pusher 304 may include at least one socket 306 for receiving and/or coupling to at least one deployment member 124a, deployment member 124b, or deployment member 124c. For example, delivery system 300 may include the socket 306 (e.g., an accommodation cavity) for receiving deployment member 124b, holding deployment member 124b within the socket 306 during implantation of device 100, and/or during de-implantation of device 100. In particular, the socket 306 may be shaped to receive an end portion 308 of deployment member 124b during deployment/implantation. To disengage the end portion 308 of device 100 from delivery system 300, the system 300 may be twisted to align the end portion 308 with aperture 310 to enable deployment member 124b to be lifted and released from delivery system 300.

The pusher 304 includes a slot 312 for receiving the actuation member 166 therethrough. In addition, the slot may be further aligned to receive actuation wire (e.g., control wire 183) therethrough. The outer sheath 302 may house the pusher 304 until the delivery system 300 is aligned to an implant site. In some examples, the device 100 may also be collapsed and housed within the outer sheath 302 until the delivery system 300 is aligned to the implant site and caused to deploy the device 100 using the pusher 304.

In some examples, the device 100 may include a frame 106 having a proximal end 108 and a distal end 110 with a longitudinal axis (L) extending therethrough. The frame may include an inner surface (e.g., inner wall 172) having a first polymeric covering 174 and an outer surface (e.g., outer wall 176) having a second polymeric covering 178. The first polymeric covering 174 may be coupled to portions of the second polymeric covering 178 to form a circumferentially sealed space 180 extending along at least a portion of frame 106. The circumferentially sealed space 180 may house at least one waist member 185 extending around an outer circumference of the outer surface 176 and encased in a space between the first polymeric covering 174 and the second polymeric covering 178.

In some examples, the frame 106 is formed from a plurality of struts spaced from the proximal end 102 to the distal end 110 (e.g., outflow end 104) and extending from each other around the frame 106 to form two or more rows of cells (e.g., cell 112, cell 114, cell 116). In such an arrangement, the first polymeric covering 174 and the second polymeric covering 178 may be heat shrunk together around the frame 106 at the proximal end (e.g., inflow end 102). In addition, a plurality of crowns 126a-126i in the frame at the proximal end 102 may be mitered (or otherwise cut) around to form cut reliefs 181a, 181b between each crown at the proximal end 102.

The cut reliefs (e.g., cut relief 181a, cut relief 181b, etc.) may include open space formed by cutting into the first polymeric covering 174 and the second polymeric covering 178 from about 0.1 millimeters to about 15 millimeters from an edge formed by the coupled first polymeric covering 174 and the second polymeric covering 178. In some examples, the cut reliefs 181a, 181b,, etc. may be shaped based on a shape of an end portion (e.g., a crown or a space between crowns) at the proximal end 102 of the frame 106. For example, the crowns (e.g., crown 126f, crown 126i, etc.) in the frame 106 at the proximal end form a basis of a shape of a plurality of cut reliefs (e.g., cut relief 181a, 181b,, etc.), formed between each crown at the proximal end. The cut reliefs 181a, 181b, etc. surrounding the end 102 may be within about 0.1 mm to about 0.3 mm from a portion of the frame 106. For example, the cut reliefs described herein may be mitered, melted, or otherwise cut into the polymeric coverings 174, 178 such that each cut relief follows a shape of the underlying frame 106 to provide a maximum amount of space between the crown top portions while maintaining a substantially sealed edge surrounding the end 102 of the frame 106.

In some examples, the frame 106 may include a plurality of adjacent struts forming a diamond shape that are spaced from the proximal end (e.g., inflow end 102) to the distal end 110 (e.g., outflow end 104) and extending from each other around the frame 106 to form two or more rows of cells (x), (y), and (z), as described elsewhere herein.

In general, portion of covering 174 may be laminated or otherwise sealed to portion of covering 178 to block blood flow through the polymeric layers 174, 178, but may leave portions open to enable a control element (e.g., control wire 183, actuation member 166, etc.) move within one or more pockets, such as space 180. The device 100 may further include an actuation member 166 (e.g., tube, wires, etc.) coupled to the frame 106. In some examples, the actuation member 166 is an elongate tube arranged substantially parallel to the longitudinal axis (L) and extending beyond the proximal end 108 of the frame 106. In some examples, a portion of the actuation member 166 may be encased between the first polymeric covering 174 and the second polymeric covering 178.

In some examples, the actuation member 166 may actuate the at least one waist member 185 to collapse the distal end 110 (e.g., outflow end 104) of the frame 106 toward a central axis (C) of the frame 106, or expand the distal end of the frame 106 away from the central axis (C), in response to an actuation of a control wire 183 (e.g., 183a, 183b) coupled to the at least one waist member 185. In some examples, the control wire 183 is further threaded through the elongate tube of the actuation member 166.

In some examples, the waist member 185 includes a plurality of eyelets 182a, 182b, etc. coupled to the frame 106 at locations around the outer circumference and arranged substantially perpendicular to the longitudinal axis (L) and within a threshold distance of the distal end 110 (e.g., outflow end 104) of the frame 106. The eyelets may be threaded with the control wire 183 to enable movement of the distal end of the frame 106 to modulate flow through the frame 106. In some examples, the eyelets 182a, etc. are each formed of shape memory alloy that may be coupled to the frame 106 at a predefined distance from the distal end 110 (e.g., outflow end 104) of the frame and around the circumference of the outer surface of the frame 106. Such eyelets 182a, etc. may be shaped brackets with a first end and a second end that is in line with the first end. The shape may be u-shaped, arched, semi-circular, circular, square, or other shape defining a through hole in which to receive a control wire threaded therethrough. The first end 184a may be coupled to a first aperture 162 defined on the outer surface of the frame 106 and the second end 184b is coupled to a second aperture 164 defined on the outer surface of the frame 106.

In some examples, the device 100 may include a skirt membrane 186 wrapped around the second polymeric covering and disposed offset from a lateral centerline at a midpoint of the frame and toward the proximal end of the frame, wherein the skirt membrane 186 is further configured to reduce blood stasis around the implantable device.

FIG. 4 is a block diagram of an example system 400 for modulating blood flow through one or more blood vessels. The system 400 may be used with any of the flow restricting devices described herein. As shown, the system 400 includes flow restriction controls 402 and at least one implantable device 404. The implantable device 404 may correspond to any of the flow restricting devices described herein.

The flow restriction controls 402 may include one or more optional sensors 406, one or more processors 408, one or more control devices 410, and one or more actuation devices 412. Optionally, the flow restriction controls may include a power source 414 that may be internal to the controls 402, internal to the implantable device 404, or external to both the flow restriction controls 402 and the implantable device 404. In some examples, the power source may be wired to flow restriction controls 402 or implantable device 404. In some examples, the power source may be remotely accessed (e.g., wirelessly) by flow restriction controls 402 or implantable device 404.

The optional sensors 406 may generally function to sense (e.g., detect) properties of the blood in which the sensor(s) are disposed within. For example, the optional sensors 406 may detect blood pressure within the blood vessel and/or any other physiological or anatomical parameters or properties of the blood or vessel. The optional sensors 406 may include one or more of an image sensor, a strain gauge, a piezoelectric sensor, a capacitance sensor, and/or a vacuum pressure sensor. In general, sensor signals from sensors 406 may be transmitted to control devices or elements described herein via a wired or wireless connection. Additionally, and optionally, the sensors 406 may utilize one or more processors 408 to transmit data to remote computing devices. The transmitted data may include sensor measurements, device position data and/or statistics, actuation events, or any other data from the system.

The processors 408 may include one or more microprocessors, microcontrollers, or the like, as described elsewhere herein. The control devices 410 may include active or passive controls including, but not limited to wires, sutures, operated switches, motor controllers, and/or antennas. In some examples, the control devices 410 may include external control devices including, but not limited to remote computers, tablets, smart phones, and/or external control devices for powering and/or controlling the flow restriction controls 402.

The actuation devices 412 may include mechanically actuating devices, electrically actuated devices, electromechanically actuated devices, or a combination thereof. For example, actuation devices 412 may include any one or more of a wire, a suture, a pull wire, a linear actuator (e.g., a pneumatic linear actuator, an electromechanical linear actuator, or a hydraulic linear actuator), a magnet or coil, etc. The power sources 414 may include, but are not limited to, battery power, wall power, magnets, induction coils, or the like.

In operation of system 400, the actuation device 412 may be coupled to the control device 410, which may manipulate or move portions of the implantable device 404 based on one or more signals received from a sensor 406. In embodiments that utilize a processor 408, the processor 408 may be communicatively coupled to sensors 406, control devices 410, actuation devices 412, power source 414, and/or implantable device 404 to actuate the implantable device 404 into a restricted blood flow state, an unrestricted blood flow state, or any position therebetween.

FIG. 5A is a schematic diagram of an example embodiment of a system 500 for modulating blood flow through a blood vessel. The system 500 may include a first magnet 506, an actuation device 508, and a control element 514. The first magnet 506 may be operatively coupled to the actuation device 508. The actuation device 508 may be operatively coupled to the control element 514 to effect movement of the control element 514. In some examples, the control element 514 is a membrane. In some examples, the control element 514 is a control wire. In some examples, the control element 514 is a catheter portion. In some examples, the control element 514 is a ring.

The system may include a control device 502 operatively coupled to a second magnet 504. The control device 502 can include a microprocessor, power source (a battery, a capacitor, wall outlet, or any other suitable power source), antenna, operated switches, and/or any other control devices. The control device 502 and second magnet 504 may be located externally, but proximal to a user. In some examples, the control device 502 and second magnet 504 may be implanted (e.g., subcutaneously, intravascularly, etc.). In some examples, both the first magnet 506 and the second magnet 504 may be permanent magnets. In some examples, the first magnet 506 is a permanent magnet and the second magnet 504 is an electromagnet.

Optionally, the system 500 may include a sensor 510 (e.g., sensor 406). The sensor 510 may sense one or more physiological or anatomical attributes and output a signal to the control device 502, which may output an activation signal to the actuation device 508 to tension or release tension in the control element 514.

The second magnet 504, although external to the user or implanted at a second location (the implantable device being at a first location), may be placed operationally proximal to the first magnet 506. By doing so, the magnetic pole orientation of the second magnet 504 influences the magnetic pole direction of the first magnet 506. For example, a magnetic gear train may be generated between the second magnet 504 and the first magnet 506, such that when the control device 502 rotates the second magnet 504, the first magnet 506 is rotated in an opposing direction. Rotating the first magnet 506 induces movement in the actuation device 508, which tensions or releases tension in the control element 514 or moves the control element 514 to a restricted or unrestricted blood flow state, respectively.

The control device 502 may receive signals from one or more optional sensors 510. Such signals may be indicative of characteristics of blood flow in the blood vessel (e.g., blood pressure). For example, when the control device 502 receives a signal indicative of a measured pressure higher than a predefined level, the control device 502 can cause the second magnet 504 to rotate. The rotation of the second magnet 504 can cause the first magnet 506 to rotate, which may actuate the actuation device 508 to move the control element 514 towards a restricted blood flow state. Further, when the control device 502 receives a signal indicative of a measured pressure lower than the predefined level, the control device 502 may cause the second magnet 504 to rotate in an opposing direction. The rotation of the second magnet 504 causes the first magnet 506 to rotate, thereby actuating the actuation device 508 to move the control element 514 into the unrestricted blood flow state.

In general, sensor signals from sensors 510 may be transmitted to control devices or elements described herein via a wired or wireless connection. Additionally, and optionally, the sensors 510 may utilize one or more processors to transmit data to remote computing devices. The transmitted data may include sensor measurements, device position data and/or statistics, actuation events, or any other data from the system.

FIG. 5B is a schematic diagram of an example embodiment of a system 550 for modulating blood flow through a blood vessel. Similar to FIG. 5A, FIG. 5B shows a magnetic gear train for manipulation of a control element 514 of an implanted device. The control device 502 and second magnet 504, as in FIG. 5A, may be located external to the user or implanted (e.g., subcutaneously, intravascularly, etc.). Similar to FIG. 5A, the first magnet 506, actuation device 508, control element 514, and optional sensor 510 may be implanted within the user. Unlike FIG. 5A, the embodiment of FIG. 5B includes an implanted (in some embodiments, implanted subcutaneously) repeater magnet 505. This repeater magnet 505 may be used to extend the operational distance between the second magnet 504 and the first magnet 506, as it is implanted at an appropriate position between the two. Additionally, the repeater magnet 505 may be used to increase the torsional force that can be applied by the magnetic gear train. Further contemplated embodiments may include a repeater module with a second power source operatively coupled to the repeater magnet 505 and capable of charging and/or powering the rotation of the repeater magnet 505. Further, the rotation direction of the second magnet 504 and first magnet 506 are now the same, not opposing one another as in the embodiment of FIG. 5A. For example, when manipulating the control element 514 towards the restricted or unrestricted blood flow states, the second magnet 504 can be rotated in the same direction as the desired direction of the first magnet 506.

### Methods

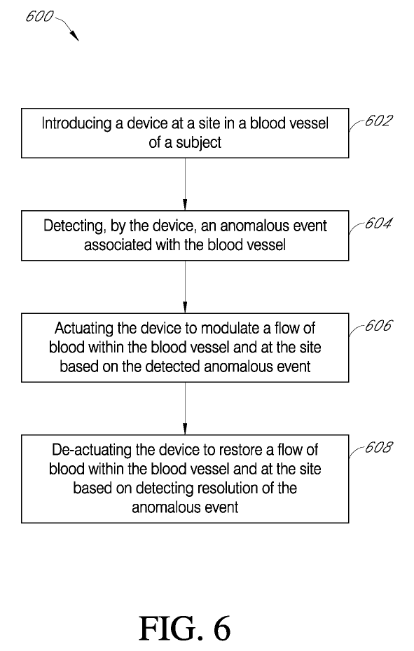

FIG. 6 is a flow diagram of an example process 600 of modulating blood flow through one or more blood vessels. The process 600 functions to reduce blood flow at a target site in a blood vessel of a heart of a subject. In some embodiments, the process 600 functions to modulate a volume of blood flowing from the blood vessel into a right atrium to modulate/decrease blood flow in order to reduce pressure to the right atrium, for example. The process 600 may be used for blood flow regulation in the vena cava, superior vena cava, or inferior vena cava, but can additionally, or alternatively, be used for any suitable applications, clinical or otherwise. In general, process 600 may be used with any of the devices described herein.

As an example, the device used with process 600 may include device 100 having a frame having an inflow end, an actuatable outflow end, and a longitudinal axis extending therethrough, an inner wall with a first polymeric covering, an outer wall with a second polymeric covering in which the first polymeric covering is coupled to portions of the second polymeric covering to form a circumferentially sealed space defined to extend around a portion of a circumference of the outer wall of the frame. In some examples, the circumferentially sealed space 180 may also extend longitudinally along at least a portion of the frame around the circumference of the outer wall. The frame may also include a skirt membrane 186 wrapped around an exterior surface of the second polymeric covering and disposed offset from a lateral centerline at a midpoint of the frame and toward the inflow end of the frame.

In some examples, the process 600 may be a method of treatment for modulating blood flow in a superior vena cava in a subject having chronic kidney disease and/or chronic heart failure. The method of treatment may include introducing a vessel occlusion device at a site in a blood vessel of a subject (block 602). For example, the devices described herein may be partially or fully housed by a frame (e.g., a stent). The frame housing the device 100, for example, may be introduced to a vessel or tissue site using a delivery system. In a coronary procedure, a catheter tip and/or catheter may be configured to pass from the right atrium into the coronary sinus to implant the device 100. For access to the venous circulation, for example, a catheter tip and/or catheter may be configured to pass from the radial artery into the superior vena cava to implant the device 100 into a portion of the superior vena cava. Further, for central venous access, a catheter tip and/or catheter may be configured to pass from the femoral vein into the inferior vena cava to implant the device 100 into a portion of the inferior vena cava.

At block 604, the method of treatment may include detecting, by the device, an anomalous event (or several events) associated with the blood vessel. For example, the process 600 may include detecting an increasing blood pressure in the right atrium. In particular, the device 100 may include one or more sensors to detect the increased blood pressure and/or the rate of increase. In some examples, the one or more sensors may be configured with a predefined blood pressure threshold level where detecting blood pressure above the predefined blood pressure threshold triggers the device to actuate and lower the blood flow to reduce pressure to the heart, for example. In some examples, the predefined threshold may pertain to a rate of pressure increase. In such examples, the one or more sensors may detect that the rate is above the predefined threshold level for rate increase and may cause the device 100 to actuate to modulate a flow of blood within the blood vessel, as shown at block 606. For example, the device 100 may be actuated based on a sensed anomalous event (or several events). Actuating the device 100 may trigger modulation of a flow of blood within the blood vessel at the device 100 site based on the detected anomalous event. For example, actuating the device 100 may cause a partial occlusion of the blood in the blood vessel at the site of the device. For example, if the device 100 is implanted into the superior vena cava of a subject having chronic kidney disease and/or chronic heart failure, the device can be actuated to modulate a volume of blood flowing from the blood vessel into a right atrium to decrease right atrial pressure.

Further, one or more sensors may be used in conjunction with any of the devices and systems herein to measure one or more physical characteristics of a patient having one of the devices implanted. For example, it may be beneficial to measure whether the patient is standing, sitting, or laying. In addition, the pressure thresholds for activating the device may be influenced by the activity of the patient. For example, it may be beneficial to realize the patient is exercising, as this would elevate pressures and may cause an adjustment in pressure thresholds. Characteristics described above may be measured by a pressure sensor in blood vessels of other portions of the body, a gyroscopic sensor for changes in angular position, an accelerometer for changes in acceleration, a heart rate sensor, a sensor measuring a size of a blood vessel, or any other sensors for measuring physical characteristics. The described characteristics, individually or in combination, may be received by a microprocessor and processed to cause changes in valve position (using an actuating device) based on the sensed characteristics.

At block 608, the method of treatment may include de-actuating the device to restore a flow of blood within the blood vessel and at the site based on detecting resolution of the anomalous event. For example, when one or more sensors of device 100 detects a resolution of the blood pressure (e.g., the blood pressure is below the threshold level), then the device 100 may trigger a de-actuation of blood flow modulation, which may function to maintain or regain a flow of blood within the blood vessel. Maintaining a flow of blood within the blood vessel may include ensuring the device 100 is held in a particular state of blood regulation such that one or more components of the device may be held stationary over time. Regaining a flow of blood within the blood vessel may include relaxing any blood occlusion components or structures such that the flow of blood may pass through the blood vessel unencumbered.

### Example implantation of Flow Modulating Devices

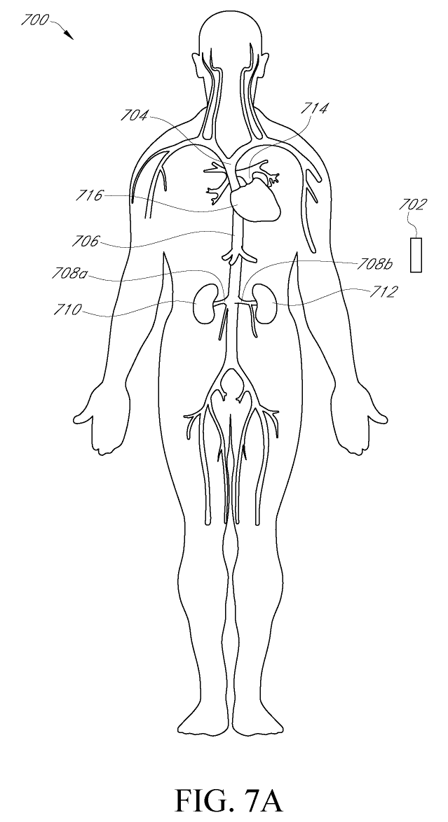

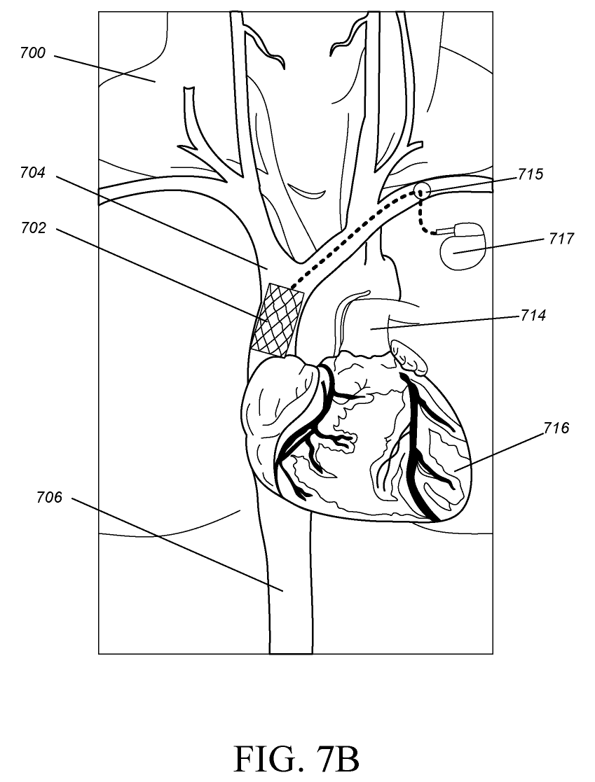

FIGS. 7A-7B illustrate schematic representations of portions of a subject 700. The flow modulating devices described herein (represented in FIG. 7A by device 702) may be introduced (e.g., implanted) in vasculature of the body. In general, the device 702 may represent any of the flow modulating devices described herein (e.g., device 100) and may include the same or similar functionality and/or structures. In some examples, the device 702 may be implanted in or near to a portion of the Superior Vena Cava (SVC) 704. In some examples, the device 702 may be implanted in or near to a portion of the Inferior Vena Cava (IVC) 706. The subject 700 is illustrated with a representation of a portion of the vasculature system to generally illustrate the SVC 704 and the IVC 706 within the subject 700. However, it is to be understood that no dimensions or relative sizes of components may be inferred from the relative sizes and dimensions of elements in the figures.

As shown in FIG. 7A, the subject 700 includes a number of vessels and organs that may circulate blood throughout the body. For example, renal veins 708a and 708b drain blood from respective right kidney 710 and left kidney 712. Renal veins 708a and 708b connect to the IVC 706. Blood from the aorta 714 flows to the IVC 706. Blood travels from the aorta 714 to the abdominal organs including the stomach (not shown), liver (not shown), spleen (not shown), pancreas (not shown), large intestines (not shown), and small intestine (not shown). Following processing of the blood by the liver, blood collects in the central vein. Blood from these central veins converges in the hepatic veins (not shown) which exit the liver and empty into the IVC 706 to be distributed to the rest of the body.

Portions of the above-recited blood circulating vessels and/or organs may be involved in splanchnic venous circulation that includes blood flow originating from the celiac, superior mesenteric, and inferior mesenteric arteries to the abdominal organs. The splanchnic venous circulation may act as a blood reservoir that can support the need for increased stressed blood volume during periods of elevated sympathetic tone, such as during exertion, to support increased cardiac output and vasodilation of peripheral vessels supporting active muscles.

Heart failure patients can have multiple comorbidities that cause excessive congestion or accumulation of blood volume in the splanchnic venous circulation. The excessive congestion or accumulation causes excess load on the heart, over-reactive fight or flight responses, poor oral medication absorption, etc. Example comorbidities can include chronic kidney disease, chronotropic incompetence, inability to increase stroke volume, and/or peripheral microvascular dysfunction. This can lead to venous congestion and/or abrupt rises in central venous pressure, pulmonary artery pressure, and/or pulmonary capillary wedge pressure. To alleviate such pressures, the blood reserves within the blood reservoir described above can be used to support the need for increased stressed blood volume during periods of elevated sympathetic tone. The flow modulating devices described herein may be used to ensure that such blood reserves within the blood reservoir can be utilized. For example, because blood flow from the splanchnic venous circulation is directed through hepatic veins and into the IVC 706, devices (as described herein) may be placed into the IVC 706 to limit blood flow to allow the splanchnic venous circulation to expand with increased blood volume. This may also allow the body to accumulate blood volume in the splanchnic venous circulation, which can maximize the downstream drop of pressure relative to upstream increase of pressure. Similarly, devices (as described herein) may be placed into the SVC 704 to limit blood flow to allow the reservoir to expand with increased blood volume. Furthermore, as shown in FIGS. 7A-7B, the flow modulating devices described herein may be placed in either the IVC 706 and/or SVC 704 to alleviate pressure in the right side of the atrium of the heart 716 and/or regulate renal venous pressure and kidney function. Another example positioning of a flow modulating device may be in the IVC below the renal veins. This positioning may have a similar effect as the SVC location, as it may allow the flow modulating device to maintain renal venous pressure, which can correlate with sustained renal function and diuresis.

As shown in FIG. 7B, the flow modulating devices described herein, represented by device 702, may be controlled by one or more flow restriction controls 717, for example as shown and described elsewhere herein and with respect to FIG. 4. The flow modulating device 702 may be implant using access site or port 715. Although access using a subclavian vein is shown in FIG. 7B, access may also be achieved using a radial artery, brachial artery, internal jugular vein, common femoral vein, or the like.

In some examples, the flow modulating device 702 (representing the devices described herein) may be used as a method of treatment to treat any combination of heart failure, chronic kidney disease, chronotropic incompetence, inability to increase stroke volume, and/or peripheral microvascular dysfunction. In addition, the flow modulating device 702 may be used as a method of treatment to regulate blood flow and therefore regulate pressure in the right atrium of the heart. Further, the flow modulating device 702 may be used as a method of treatment to improve function of the kidneys and/or heart in patients having reduced kidney function due to pressure in the venous system.

For example, any of the implantable devices and/or systems described herein may be configured to modulate a volume of blood flowing from a superior vena cava into a right atrium to decrease right atrial pressure. Further for example, any of the implantable devices and/or systems described herein may be used to perform a method including restricting blood flow within a blood vessel. Still further for example, any of the implantable devices and/or systems described herein may be used to perform a method of treatment for a subject having one or both of: congestive heart failure or chronic kidney disease. The method may include restricting blood flow within the blood vessel.

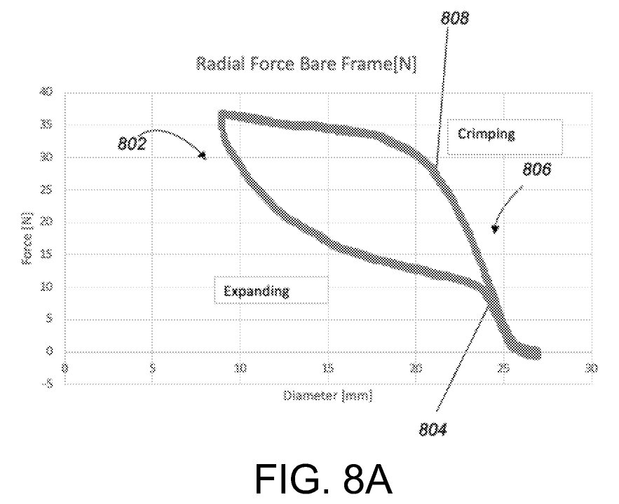

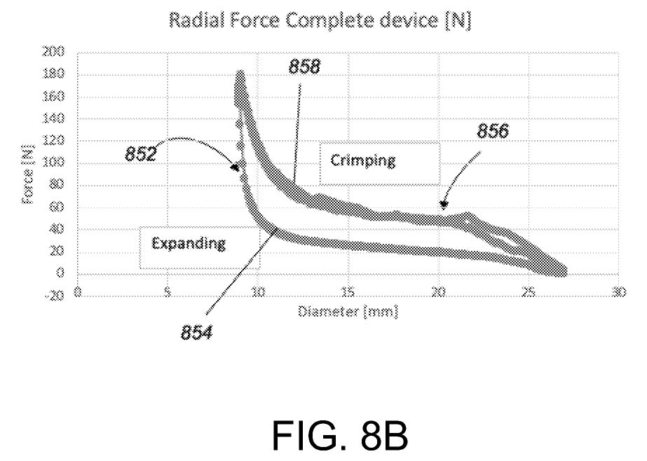

FIG. 8A illustrates example radial force plot curves measured during expanding and crimping processes occurring over time for the devices described herein without covered portions. The measurements were performed to capture radial force (e.g., chronic outward force) from device 100 (without the first polymeric layer 174, the second polymeric layer 178, or the skirt membrane 186) during crimping and expanding processes. Device 100 may be self-expanding.

A first curve 802 depicts the radial force of the device 100 during an expansion process. As the diameter of the device 100 is increased during the expansion process, the radial force decreases at a substantially constant rate until the diameter approaches about 25 mm, at point 804. The radial force begins to fall more rapidly until the diameter approaches about 27 mm.

A second curve 806 depicts the radial force of the device 100 during a crimping process. As the device 100 is crimped, the radial force substantially maintains a force between about 37 N and about 34 N and falls off at a first rate until about point 808, where the radial force falls off at a steeper, second rate. The expansion process shown by curve 802 and the crimping process shown by curve 806 represent expansion forces or crimping forces relevant to loading of the device 100 with respect to a sheath (e.g., delivery tube retaining the device 100). For example, the radial force shown represents a fixed device 100 in a vessel without modulating the end portion (e.g., distal end 110).

FIG. 8B illustrates example radial force plot curves measured during expanding and crimping processes occurring over time for the devices described herein with covered portions. The measurements were performed to capture radial force from device 100 (including the first polymeric layer 174, the second polymeric layer 178, and the skirt membrane 186) during crimping and expanding processes. A first curve 852 depicts the radial force of the device 100 during an expansion process. The first curve 852 drops in force steeply until about 40 N, at point 854, and subsequently falls off over time from about 40 N to about 0 N as the diameter of the device 100 is increased. A second curve 856 depicts the radial force of the device 100 during a crimping process. The second curve 856 follows a similar shape as the first curve 852 with a corresponding point to point 854 occurring at point 858 at about 60 N.

The expansion process shown by curve 852 and the crimping process shown by curve 856 represent expansion forces or crimping forces relevant to loading of the device 100 with respect to a sheath (e.g., delivery tube retaining the device 100). For example, the radial force shown represents a fixed device 100 in a vessel without modulating the end portion (e.g., distal end 110).

As used herein, the term “active” with respect to blood flow management may represent operations carried out by the devices described herein using power or controller induced movement. For example, actively moving a portion of the devices described herein may include the use of battery power, wall outlet power, magnetic field induction, electromagnetic field induction, magnetic polarization, a piston-based system, a valve based system (e.g., with a manifold), hydraulics, pneumatics, optical actuators, thermal actuators, and/or other actuator using electrical or inductive power.

In some examples, an active control mechanism may include a microcontroller and/or a power source implanted with or integrated with the flow management device. Alternatively, or additionally, an active control mechanism can include a microcontroller and/or a power source in a remote control device, external to the body, or in an implanted remote device (e.g., subcutaneously, intravascularly, etc.), for example. The remote control device may be in wireless communication with the implanted device or connected to the implanted device through one or more leads.

In any of the embodiments described herein, an active mechanism may include an actuator (e.g., a linear actuator) coupled to a control element of the flow management device. The linear actuator tensions the control element to position the valve of the flow management device in a restricted blood flow state. Alternatively, the linear actuator releases tension in the control element to position a valve, a membrane, or other material in an unrestricted blood flow state. The tensioning and releasing of tension on the control element may be based on a predefined set of parameters or based on a sensed attribute of the blood vessel in which the flow management device is implanted. For example, the sensed attribute may be sensed by a sensor. The sensor may be coupled to the flow management device, a remote control device, or otherwise in wireless or electrical communication with a flow management system. The sensor can be a strain gauge, a piezoelectric sensor, a capacitance sensor, or a vacuum pressure sensor, such that the sensor senses a pressure in the blood vessel.

In any of the embodiments described herein, the linear actuator is an electromechanical linear actuator having a first magnet that, when caused to rotate by another magnet or actuator, causes a nut to rotate on a lead screw, the nut being coupled to the control element. A second magnet in a control device may cause rotation of the first magnet, for example by changing its magnetic field pole direction. In some embodiments, a repeater magnet (with or without its own power source) is positioned between the first magnet and the second magnet, for example in cases where the first magnet is beyond a threshold distance from the second magnet.

In any of the embodiments described herein, the linear actuator is a pneumatic linear actuator having a piston coupled to the control element. Injecting compressed gas moves the piston to tension the control element to move the valve into a restricted blood flow state and venting the compressed gas releases tension in the control element to move the valve to an unrestricted blood flow state.

In any of the embodiments described herein, the linear actuator is a hydraulic linear actuator having a piston coupled to the control element. Injecting liquid moves the piston to tension the control element to move the valve into a restricted blood flow state and venting the liquid releases tension in the control element to move the valve to an unrestricted blood flow state.

In any of the embodiments described herein, the linear actuator is a thermal linear actuator having a piston coupled to the control element. For example, decreasing a temperature of a thermal sensitive fluid (e.g., via a heat source, changes in body temperature, etc.) causes the piston to compress the fluid to tension the control element to move the valve into the restricted blood flow state. Alternatively, increasing the temperature of the thermal sensitive fluid causes the piston to decompress the fluid to release tension in the control element to move the valve to the unrestricted blood flow state.

As used herein, the term “passive” with respect to blood flow management may represent operations carried out by the devices described herein using passively induced movement. For example, passively moving a portion of the devices described herein may include the use of manual pull wires (e.g., sutures, actuation wires/cords, actuation members, tubes, etc.), anatomy responses (e.g., changes in vessel inner diameter, intra-vessel pressure, etc.), blood movement, or the like.

Any of the implantable or flow modulating devices described herein may be coated with a polymer (e.g., silicones, poly(urethanes), poly(acrylates), or copolymers such as poly(ethylene vinyl acetate), a drug (e.g., heparin, pro-endothelialization drugs, anti-thrombogenic drug, etc.), a textile (e.g., woven, knitted, nonwoven, or braided), tissue (e.g., bovine pericardium, equine pericardium, porcine vena cava, etc.), or a combination thereof. Woven and knitted fabrics may be made from poly(ethylene terephthalate), while the nonwoven fabrics may be made from expanded poly(tetrafluoroethylene). Some textiles may also or alternatively include silk or silk-based materials.

Further, any of the pull wires, sutures, frames/stents, or actuation wires described herein may include silk, silk-based materials, nylon, synthetic polymer materials (e.g., silicone, polydioxanone, polyglycolic acid, polyglyconate, polylactic acid, etc.), natural materials (e.g., purified catgut, collagen, sheep intestines, cow intestines, etc.), metal (e.g., Nitinol®, palladium, gold and their alloys, etc.), or a combination thereof.

The flow modulating devices described herein may be part of (or installed within) a stent (e.g., a frame and/or a braid). The stent may represent a frame or outer frame that provides a support structure for the flow modulating devices when the stent is implanted into a blood vessel. The frame/outer frame may be a self-expanding frame or a balloon-expandable frame. In general, any type of stent may be used with the flow modulating devices. Example stents may include, but are not limited to, bare metal stents, coated stents, drug-eluting stents, biodegradable stents, balloon expandable stents, and self-expandable stents.

The stents described herein may be configured to house all or a portion of the flow modulating devices described herein. Such stents may include an assembly with struts (e.g., strut members) members interconnected by joints that form a series of linked mechanisms that result in a hollow tube-shaped element. The stents may be positioned and/or repositioned within a blood vessel to introduce or remove flow modulating devices or device members including, but not limited, to valving, control elements, balloons, flexible members, rigid members, adjustment mechanisms, sensors, coils, wires, and/or magnets. One or more of such device members may be actuated to modify stent shape (or device member shape) for purposes of modifying a flow of fluid through the vessel associated with the implanted stent. Moreover, the stents described herein may partially or fully surround a flow modulating device. For example, a stent or stent portion may surround a portion of a flow modulating device to ensure the device remains in a specified position in a blood vessel. In some examples, the stent surrounds the flow modulating device entirely. In some examples, the stent surrounds the flow modulating device and further continues beyond one or both ends of the device.

The stents described herein may represent an outer frame. The outer frame may have a form and structure that varies. For example, the struts and/or joints may form a mesh-like structure. The struts may be interconnected in such a way as to form a shaped pattern of cells. For example, any number of struts may form a ring of the stent (e.g., frame) such that the struts are connected by any number of crowns. Any number of rings may form a body of the stent, and the rings may be connected by any number of bridges. Example cell shapes may include, but are not limited to diamond, square, rectangle, triangle, oval, ganglion, or any combination thereof. In some examples, the cells may be evenly shaped and distributed from a first end of the stent to a second end of the stent. In some examples, the cells may include a number of struts interconnected in such a way that when the stent expands radially, one or more of the cells become longitudinally shorter. Similarly, when the stent constricts radially, one or more of the cells become longitudinally longer.

Constricting portions of the stents described herein may result in an outer frame woven tighter than other portions of the stent that are not constricted. The constriction may push against one or more portions of the flow modulating devices described herein to narrow a pathway through the frame or outer frame and/or to trigger the flow modulating device to begin or end constriction. Similarly, expanding portions of the stents described herein may result in an outer frame woven looser than other portions of the stent that are not expanded. The expansion may release one or more portions of the flow modulating devices described herein to widen a pathway through the frame or outer frame and/or to trigger the flow modulating device to begin or end constriction.

The flow modulating devices described herein may be introduced to a vessel or tissue site using a delivery system. For example, such delivery systems may be used to position catheter tips and/or catheters in various portions of a target vasculature. A delivery system may include a delivery catheter having a pusherwire or the like disposed therein. The pusherwire may be configured to deploy any of the devices described herein, for example by urging the device out of a distal end of the catheter and either actively expanding the device or allowing the device to passively expand once it is no longer constrained by a lumen of the catheter. Any of the devices described herein may be crimped or otherwise compressed such that a cross-sectional area of the device is sized and/or shaped to be delivered through a lumen of a catheter. In some examples, the crimped or compressed device may be transferred to the delivery system using a transfer sheath, or the like. A delivery system can access the vasculature through an access site, such as a radial artery, brachial artery, internal jugular vein, common femoral vein, subclavian veins, or the like.

For example, in a coronary procedure, a catheter tip and/or catheter may be configured to pass from the right atrium into the coronary sinus. For access to the venous circulation, for example, a catheter tip and/or catheter may be configured to pass from the radial artery into the superior vena cava. Further, for central venous access, a catheter tip and/or catheter may be configured to pass from the femoral vein into the inferior vena cava.

In some examples, the delivery system may include a trocar or other suitable delivery device used for implanting devices subcutaneously, for example control devices for controlling activation of any of the flow modulating devices described herein. As described elsewhere herein, various control systems may include an implanted remote device that is configured to transmit control signals to a flow modulating device disposed in the vasculature. The control signals may include signals transmitted wirelessly, through a wired connection (e.g., leads), or via magnetic field induction, electromagnetic field induction, or magnetic polarization.

However, it will be understood that the delivery system can refer or generally apply to positioning of catheter tips and/or catheters from a first body chamber or lumen into a second body chamber or lumen, where the catheter tips and/or catheters may be bent when positioned from the first body chamber or lumen into the second body chamber or lumen. A body chamber or lumen can refer to any one of a number of fluid channels, blood vessels (e.g., superior vena cava, inferior vena cava, renal artery, renal vein, etc.), and/or organ chambers (e.g., heart chambers). Additionally, reference herein to “catheters,” “tubes,” “sheaths,” “steerable sheaths,” and/or “steerable catheters” can refer or apply generally to any type of elongate tubular delivery device including an inner lumen configured to slidably receive instrumentation, such as for positioning within an atrium, coronary sinus, superior vena cava, or inferior vena cava, including for example delivery catheters, cannulas, and/or trocars. It will be understood that other types of medical implant devices and/or procedures can be delivered to the coronary sinus, superior vena cava, inferior vena cava, etc. using a delivery system as described herein, including for example ablation procedures, drug delivery, and/or placement of actuator leads.

Described herein are various example medical implants and/or delivery methods. Some examples described herein may be used in combination and/or may be used independently.

Provided below is a list of examples, each of which may include aspects of any of the other examples disclosed herein. Furthermore, aspects of any example described above may be implemented in any of the numbered examples provided below.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Example 1. An implantable device for modulating blood flow through a blood vessel, the implantable device comprising: a frame having an inflow end, an actuatable outflow end, and a longitudinal axis extending therethrough, the frame comprising: an inner wall with a first polymeric covering: an outer wall with a second polymeric covering, wherein the first polymeric covering is coupled to portions of the second polymeric covering to form a circumferentially sealed space defined to extend around a portion of a circumference of the outer wall of the frame; and a skirt membrane wrapped around an exterior surface of the second polymeric covering and disposed offset from a lateral centerline at a midpoint of the frame and toward the inflow end of the frame.

Example 2. The implantable device of any of the preceding examples, but particularly example 1, wherein an inflow perimeter of the skirt membrane ends within a threshold distance of the inflow end of the frame.

Example 3. The implantable device of any of the preceding examples, but particularly example 1, wherein the frame is formed from a plurality of struts spaced from the inflow end to the outflow end and extending from each other around the frame to form two or more rows of cells, wherein: the first polymeric covering and the second polymeric covering are heat shrunk together around the frame at the inflow end, and a plurality of crowns in the frame at the inflow end are mitered around to form cut reliefs between each crown at the inflow end.

Example 4. The implantable device of any of the preceding examples, but particularly example 3, wherein the cut reliefs comprise open space formed by cutting into the first polymeric covering and the second polymeric covering from about 0.1 millimeter to about 15 millimeters from an edge formed by the coupled first polymeric covering and the second polymeric covering.

Example 5. The implantable device of any of the preceding examples, but particularly example 4, wherein the cut reliefs are shaped based on a shape of an end portion of the inflow end of the frame.

Example 6. The implantable device of any of the preceding examples, but particularly example 1, further comprising: a waist member disposed around the outer wall of the frame, and wherein the circumferentially sealed space is defined to extend around the waist member.

Example 7. The implantable device of any of the preceding examples, but particularly example 6, further comprising: an actuation member coupled to the frame between the first polymeric covering and the second polymeric covering, the actuation member being configured to actuate the waist member to flexibly collapse the outflow end of the frame toward a central axis of the frame, or expand the outflow end of the frame away from the central axis, in response to actuation of a control wire coupled to the waist member and the actuation member.

Example 8. The implantable device of any of the preceding examples, but particularly example 7, further comprising a power source coupled to the control wire and configured to actuate the control wire, the power source comprising a battery.

Example 9. The implantable device of any of the preceding examples, but particularly example 7, wherein the actuation member comprises or is coupled to: an actuator coupled to the control wire of the implantable device, and a first magnet configured to induce rotation of the actuator; and a control device communicatively coupled to the actuator, wherein the control device comprises a second magnet configured to generate a changing magnetic field pole direction to cause rotation of the first magnet.

Example 10. The implantable device of any of the preceding examples, but particularly example 9, wherein: the actuation member is configured to send a first signal to the control wire to activate application of tension to the control wire; and the actuation member is configured to send a second signal to the control wire to activate release of the tension from the control wire.

Example 11. The implantable device of any of the preceding examples, but particularly example 9, wherein the actuation member is further configured to: cause rotation of the second magnet in a first direction to induce rotation of the first magnet, thereby causing the actuator to tension the control wire to cause the outflow end to radially collapse inward; and cause rotation of the second magnet in a second direction to induce rotation of the first magnet, thereby causing the actuator to release tension in the control wire to cause the outflow end to radially open.

Example 12. The implantable device of any of the preceding examples, but particularly example 9, wherein the control device is implanted subcutaneously.

Example 13. The implantable device of any of the preceding examples, but particularly example 7, wherein the waist member comprises a plurality of eyelets coupled to the frame at locations around the circumference and arranged substantially perpendicular to the longitudinal axis and within a threshold distance of the outflow end of the frame, the plurality of eyelets being threaded with the control wire.

Example 14. The implantable device of any of the preceding examples, but particularly example 13, wherein the plurality of eyelets are each: formed of shape memory alloy configured to be coupled to the frame at a predefined distance from the outflow end of the frame and around the circumference; and a plurality of brackets having a first end and a second end, wherein the first end is coupled to a first aperture defined on the frame and the second end is coupled to a second aperture defined on the frame, the first aperture and the second aperture arranged along the longitudinal axis of the frame.

Example 15. The implantable device of any of the preceding examples, but particularly example 7, wherein the actuation member is an elongate tube arranged substantially parallel to the longitudinal axis and extending beyond the inflow end of the frame, and wherein the control wire is further threaded through the elongate tube.

Example 16. The implantable device of any of the preceding examples, but particularly example 7, wherein the frame is formed from a plurality of struts spaced from the inflow end to the outflow end and extending from each other around the frame to form two or more rows of cells, wherein at least one cell comprises a first crown adjacent to a first set of apertures and a second crown adjacent to a second set of apertures, the first crown being opposite the second crown along the longitudinal axis of the frame.

Example 17. The implantable device of any of the preceding examples, but particularly example 16, wherein the first set of apertures is configured to receive a first support to hold a first portion of the actuation member and the second set of apertures is configured to receive a second support to hold a second portion of the actuation member.

Example 18. The implantable device of any of the preceding examples, but particularly example 16, further comprising: a first detent to inhibit movement of the actuation member beyond a first threshold distance toward the inflow end of the frame; and a second detent to inhibit movement of the actuation member beyond a second threshold distance toward the outflow end of the frame.

Example 19. The implantable device of any of the preceding examples, but particularly example 18, further comprising: a first support to hold a first portion of the actuation member and a second support to hold a second portion of the actuation member, wherein the first support is aligned on the frame to be contained within a first groove of the frame, and the second support is aligned on the frame to be contained within a second groove of the frame, the first groove and the second groove being parallel and along the longitudinal axis.

Example 20. The implantable device of any of the preceding examples, but particularly example 1, wherein: the first polymeric covering and the second polymeric covering comprise thermoplastic polyurethane or polyolefin; and coupling the first polymeric covering to the portions of the second polymeric covering comprises laminating the portions of the second polymeric covering to the first polymeric covering.

Example 21. The implantable device of any of the preceding examples, but particularly example 1, wherein: the blood vessel is a superior vena cava; and the skirt membrane is configured to seal an entrance to at least one additional blood vessel branching from the superior vena cava when the implantable device is implanted in the superior vena cava.

Example 22. The implantable device of any of the preceding examples, but particularly example 21, wherein the at least one additional blood vessel is an azygos junction.

Example 23. The implantable device of any of the preceding examples, but particularly example 1, wherein the skirt membrane is further configured to reduce blood stasis around the implantable device.

Example 24. The implantable device of any of the preceding examples, but particularly example 1, wherein the frame is: substantially tubular-shaped with a substantially annular cross section at the inflow end and a substantially star-shaped or polygon-shaped cross section at the outflow end when the implantable device is configured in an unrestricted blood flow state; and adjustable to form a cinched portion at the outflow end, the cinching resulting in reversibly reducing or closing the substantially star-shaped or polygon-shaped cross section at the outflow end when the implantable device is configured in a restricted or partially restricted blood flow state.

Example 25. The implantable device of any of the preceding examples, but particularly example 1, wherein the outflow end is adjustable to a plurality of positions between expanded and collapsed, the plurality of positions including at least: an expanded position configured to allow the blood flow through the blood vessel; a partially expanded position configured to partially occlude the blood vessel; and a collapsed position configured to block the outflow end to occlude the blood vessel.

Example 26. An implantable device for modulating blood flow through a blood vessel, the implantable device comprising: a frame having a proximal end and a distal end with a longitudinal axis extending therethrough, the frame comprising: an inner surface having a first polymeric covering; an outer surface having a second polymeric covering, wherein the first polymeric covering is coupled to portions of the second polymeric covering to form a circumferentially sealed space extending along at least a portion of frame, the circumferentially sealed space housing at least one waist member extending around an outer circumference of the outer surface and encased in a space between the first polymeric covering and the second polymeric covering; and an actuation member coupled to the frame and configured to actuate the at least one waist member to collapse the distal end of the frame toward a central axis of the frame, or expand the distal end of the frame away from the central axis, in response to an actuation of a control wire coupled to the at least one waist member.

Example 27. The implantable device of any of the preceding examples, but particularly example 26, wherein the at least one waist member comprises a plurality of eyelets coupled to the frame at locations around the outer circumference and arranged substantially perpendicular to the longitudinal axis and within a threshold distance of the distal end of the frame, the plurality of eyelets being threaded with the control wire.

Example 28. The implantable device of any of the preceding examples, but particularly example 27, wherein the plurality of eyelets are each: formed of shape memory alloy configured to be coupled to the frame at a predefined distance from the distal end of the frame and around the circumference of the outer surface of the frame; and a plurality of brackets having a first end and a second end, wherein the first end is coupled to a first aperture defined on the outer surface of the frame and the second end is coupled to a second aperture defined on the outer surface of the frame, the first aperture and the second aperture being along the longitudinal axis of the frame.

Example 29. The implantable device of any of the preceding examples, but particularly example 26, wherein the actuation member is an elongate tube arranged substantially parallel to the longitudinal axis , and wherein the control wire is further threaded through the elongate tube.

Example 30. The implantable device of any of the preceding examples, but particularly example 29, wherein a portion of the actuation member is encased between the first polymeric covering and the second polymeric covering.

Example 31. The implantable device of any of the preceding examples, but particularly example 26, wherein the frame is formed from a plurality of struts spaced from the proximal end to the distal end and extending from each other around the frame to form two or more rows of cells, wherein: the first polymeric covering and the second polymeric covering are heat shrunk together around the frame at the proximal end, and a plurality of crowns in the frame at the proximal end form a basis of a shape of a plurality of cut reliefs formed between each crown at the proximal end.

Example 32. The implantable device of any of the preceding examples, but particularly example 31, wherein the cut reliefs comprise open space formed by cutting into the first polymeric covering and the second polymeric covering from about 0.1 millimeters to about 15 millimeters from an edge formed by the coupled first polymeric covering and the second polymeric covering.

Example 33. The implantable device of any of the preceding examples, but particularly example 32, wherein the cut reliefs are shaped based on a shape of an end portion at the proximal end of the frame.

Example 34. The implantable device of any of the preceding examples, but particularly example 26, wherein: the first polymeric covering and the second polymeric covering comprise thermoplastic polyurethane or polyolefin; and coupling the first polymeric covering to portions of the second polymeric covering comprises laminating the portions of the second polymeric covering to the first polymeric covering.

Example 35. The implantable device of any of the preceding examples, but particularly example 26, wherein the central axis of the expandable frame is substantially parallel to the longitudinal axis.

Example 36. The implantable device of any of the preceding examples, but particularly example 26, further comprising: a skirt membrane wrapped around the second polymeric covering and disposed offset from a lateral centerline at a midpoint of the frame and toward the proximal end of the frame, wherein the skirt membrane is further configured to reduce blood stasis around the implantable device.

Example 37. The implantable device of any of the preceding examples, but particularly example 36, wherein the skirt membrane is configured to be at least partially incorporated into an inner wall of the blood vessel.

Example 38. The implantable device of any of the preceding examples, but particularly example 36, wherein: the blood vessel is a superior vena cava; and the skirt membrane is configured to seal an entrance to at least one additional blood vessel branching from the superior vena cava when the implantable device is implanted in the superior vena cava.

Example 39. The implantable device of any of the preceding examples, but particularly example 38, wherein the at least one additional blood vessel is an azygos junction.

Example 40. The implantable device of any of the preceding examples, but particularly example 26, wherein the frame comprises one or more bulbous-ended deployment members coupled to a portion of the frame at the proximal end.

Example 41. The implantable device of any of the preceding examples, but particularly example 26, wherein the frame comprises a plurality of adjacent struts forming a diamond shape that are spaced from the proximal end to the distal end and extending from each other around the frame to form two or more rows of cells.

Example 42. The implantable device of any of the preceding examples, but particularly example 41, wherein the two or more rows of cells comprise: a first row at the distal end of the frame, the first row having cells with an elongated diamond shape, each cell being formed of a first plurality of struts of a first length and first width, the first row terminating in a plurality of crowns configured to move radially inward toward a central axis of the frame and move radially outward away from the central axis; and a second row extending from the first row longitudinally toward the proximal end of the frame, the second row having cells with an elongated diamond shape formed of a second plurality of struts of a second length and a second width.

Example 43. The implantable device of any of the preceding examples, but particularly example 42, wherein the first length is greater than the second length and the first width is less than the second width.

Example 44. The implantable device of any of the preceding examples, but particularly example 42, wherein at least one cell in the second row comprises a first crown having a first set of apertures and a second crown having a second set of apertures, the first crown being opposite the second crown, wherein the first set of apertures is configured to receive a first bracket to hold a first portion of the actuation member and the second set of apertures is configured to receive a second bracket to hold a second portion of the actuation member.

Example 45. The implantable device of any of the preceding examples, but particularly example 42, wherein the two or more rows of cells further comprise: a third row extending longitudinally from the second row and to the proximal end, the third row formed of a third plurality of struts of a third length, wherein at least a portion of the third plurality of struts terminate in a deployment member.

Example 46. The implantable device of any of the preceding examples, but particularly example 45, wherein each deployment member has a bulbous end configured to be received in an accommodation cavity of a delivery device and to be released to cause implantation of the implantable device into the blood vessel.

Example 47. The implantable device of any of the preceding examples, but particularly example 42, wherein the cells of the first row further include at a midpoint, a plurality of joints, and each of the plurality of joints comprise a first aperture and a second aperture, the first and the second aperture being along the longitudinal axis of the frame.

Example 48. The implantable device of any of the preceding examples, but particularly example 47, wherein each first aperture and each second aperture are arranged to receive a wire configured to receive the control wire therethrough.

Example 49. The implantable device of any of the preceding examples, but particularly example 42, wherein the first plurality of struts in the first row is configured to provide a first radial stiffness and the second plurality of struts in the second row is configured to provide a second radial stiffness, the first radial stiffness being less than the second radial stiffness.

Example 50. The implantable device of any of the preceding examples, but particularly example 26, wherein actuating the control wire results in configuring the implantable device in an unrestricted blood flow state or a restricted blood flow state, wherein: the unrestricted blood flow state corresponds to the distal end radially expanding away from the central axis of the frame to allow blood flow through the blood vessel; and the restricted blood flow state corresponds to the distal end radially collapsing toward the central axis of the frame to reduce blood flow through the blood vessel.

Example 51. The implantable device of any of the preceding examples, but particularly example 26, wherein the distal end is adjustable to a plurality of positions between expanded and collapsed, the plurality of positions including at least: an expanded position configured to allow the blood flow through the blood vessel; a partially expanded position configured to partially occlude the blood vessel; and a collapsed position configured to block the distal end to occlude the blood vessel.

Example 52. The implantable device of any of the preceding examples, but particularly example 26, wherein the frame is: substantially tubular-shaped with a substantially annular cross section at the proximal end and a substantially star-shaped or polygon-shaped cross section at the distal end when the implantable device is configured in an unrestricted blood flow state; and adjustable to form a cinched portion at the distal end, the cinching resulting in reversibly reducing or closing the substantially star-shaped or polygon-shaped cross section at the distal end when the implantable device is configured in a restricted or partially restricted blood flow state.

Example 53. The implantable device of any of the preceding examples, but particularly example 26, wherein the blood vessel comprises a vena cava, a superior vena cava, or an inferior vena cava.

Example 54. The implantable device of any of the preceding examples, but particularly example 26, further comprising a power source coupled to the control wire, the power source comprising a battery.

Example 55. The implantable device of any of the preceding examples, but particularly example 26, wherein the actuation member comprises or is coupled to: an actuator coupled to the control wire of the implantable device, and a first magnet configured to induce rotation of the actuator; and a control device communicatively coupled to the actuator, wherein the control device comprises a second magnet configured to generate a changing magnetic field pole direction to cause rotation of the first magnet.

Example 56. The implantable device of any of the preceding examples, but particularly example 55, wherein: the actuation member is configured to send a first signal to the control wire to activate application of tension to the control wire; and the actuation member is configured to send a second signal to the control wire to activate release of the tension from the control wire.

Example 57. The implantable device of any of the preceding examples, but particularly example 55, wherein the actuation member is further configured to: cause rotation of the second magnet in a first direction to induce rotation of the first magnet, thereby causing the actuator to tension the control wire to cause the distal end to radially collapse inward; and cause rotation of the second magnet in a second direction to induce rotation of the first magnet, thereby causing the actuator to release tension in the control wire to cause the distal end to radially open.

Example 58. The implantable device of any of the preceding examples, but particularly example 55, wherein the control device is implanted subcutaneously.

Example 59. A method of treatment for reducing blood flow at a target site in a blood vessel of a heart of a subject, the method comprising: introducing a device in the blood vessel, the device comprising: a frame having an inflow end, an actuatable outflow end, and a longitudinal axis extending therethrough, the frame comprising: an inner wall with a first polymeric covering: an outer wall with a second polymeric covering, wherein the first polymeric covering is coupled to portions of the second polymeric covering to form a circumferentially sealed space defined to extend around a portion of a circumference of the outer wall of the frame; and a skirt membrane wrapped around an exterior surface of the second polymeric covering and disposed offset from a lateral centerline at a midpoint of the frame and toward the inflow end of the frame; and actuating the device to modulate a flow of blood within the blood vessel.

Example 60. The method of any of the preceding examples, but particularly example 59, further comprising: de-actuating the device to maintain or regain the flow of blood through the blood vessel.

Example 61. The method of any of the preceding examples, but particularly example 59, wherein the target site includes a portion of a superior vena cava (SVC) of a subject in which the device is implanted, or a portion of an inferior vena cava (IVC) of the subject in which the device is implanted.

Example 62. The method of any of the preceding examples, but particularly example 59, wherein actuating the device causes a partial occlusion of blood in the blood vessel.

Example 63. The method of any of the preceding examples, but particularly example 59, wherein the blood vessel is a superior vena cava and the device is configured to be implanted in a portion of the superior vena cava of a subject having chronic kidney disease and chronic heart failure; and the method further comprises modulating a volume of blood flowing from the blood vessel into a right atrium to decrease right atrial pressure.

Example 64. An implantable device for dynamically occluding a blood vessel, the implantable device comprising: a tubular frame having a lumen extended therethrough; a sealing element disposed along an exterior of the frame and along an interior of the frame opposite the exterior of the frame, the sealing element being configured to seal portions of the frame between the exterior and the interior of the frame; and an actuation member coupled to the frame and configured to alter a diameter of the frame to modulate blood flow through the lumen.

Example 65. The implantable device of any of the preceding examples, but particularly example 64, wherein the actuation member is at least partially sealed within the sealing element.

Example 66. The implantable device of any of the preceding examples, but particularly example 64, wherein the actuation member is configured to actuate a portion of an outflow end of the frame to alter the diameter of the frame.

Example 67. The implantable device of any of the preceding examples, but particularly example 64, wherein the actuation member is configured to alter the diameter of the frame responsive to receiving a signal from a pressure sensor coupled to the frame.

Example 68. The implantable device of any of the preceding examples, but particularly example 67, wherein the pressure sensor is coupled to a processor adapted to improve heart function by causing actuation of the actuation member responsive to receiving the signal from the pressure sensor.

Example 69. The implantable device of any of the preceding examples, but particularly example 64, further comprising: a waist member disposed along a portion of the frame and within the sealing element, the waist member being configured to flexibly collapse an outflow end of the frame or expand the outflow end of the frame, in response to actuation of a control wire coupled to the waist member and the actuation member.

Example 70. The implantable device of any of the preceding examples, but particularly example 69, wherein the actuation member is controlled by a power source and coupled to the control wire.

Example 71. The implantable device of any of the preceding examples, but particularly example 70, wherein: the actuation member is configured to send a first signal to the control wire to activate application of tension to the control wire; and the actuation member is configured to send a second signal to the control wire to activate release of the tension from the control wire.

Example 72. The implantable device of any of the preceding examples, but particularly example 71, wherein the actuation member extends around a periphery of the frame.

Example 73. The implantable device of any of the preceding examples, but particularly example 64, wherein the blood vessel is a vein and the frame is sized for implantation in the vein.

Example 74. The implantable device of any of the preceding examples, but particularly example 64, wherein the blood vessel is a vena cava and the frame is sized for implantation in the vena cava.

Example 75. The implantable device of any of the preceding examples, but particularly example 64, wherein the frame is composed substantially of Nitinol.

Example 76. The implantable device of any of the preceding examples, but particularly example 64, wherein the sealing element is substantially composed of polymer and comprises: a first covering disposed along an exterior of the frame; and a second covering disposed along an interior of the frame.

Example 77. The implantable device of any of the preceding examples, but particularly example 64, wherein the frame has a diameter of about 3 millimeters to about 6 millimeters.

Example 78. The implantable device of any of the preceding examples, but particularly example 64, wherein the tubular frame comprises an outflow end configured to adjust to a plurality of positions between expanded and collapsed, the plurality of positions including at least: an expanded position configured to allow the blood flow through the blood vessel; a partially expanded position configured to partially occlude the blood vessel; and a collapsed position configured to block the outflow end to occlude the blood vessel.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It should be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

The systems and methods of the embodiments and variations described herein can be embodied and/or implemented at least in part as a machine configured to receive a computer-readable medium storing computer-readable instructions. The instructions may be executed by computer-executable components integrated or in communication with the system and one or more portions of the processor on or in communication with the control device and/or computing device. The computer-readable medium can be stored on any suitable computer-readable media such as RAMs, ROMs, flash memory, EEPROMs, optical devices (e.g., CD or DVD), hard drives, floppy drives, or any suitable device. The computer-executable component is preferably a general or application-specific processor, but any suitable dedicated hardware or hardware/firmware combination can alternatively or additionally execute the instructions.

As used in the description and claims, the singular form “a”, “an” and “the” include both singular and plural references unless the context clearly dictates otherwise. For example, the term “projection” may include, and is contemplated to include, a plurality of projections. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by ( + ) or ( - ) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An implantable device for modulating blood flow through a blood vessel, the implantable device comprising:
a frame having an inflow end and an outflow end with a central longitudinal axis extending therethrough;
a first polymeric covering disposed on an inner surface of the frame;
a second polymeric covering disposed on an outer surface of the frame, wherein the first polymeric covering is coupled to portions of the second polymeric covering to form a circumferentially sealed space extending along at least a portion of frame, the circumferentially sealed space housing at least one waist member extending around an outer circumference of the outer surface and encased in a space between the first polymeric covering and the second polymeric covering; and
an actuation member coupled to the frame and configured to actuate the at least one waist member to collapse the outflow end of the frame toward the longitudinal axis of the frame, or expand the outflow end of the frame away from the longitudinal axis, in response to an actuation of a control wire coupled to the at least one waist member.

2. The implantable device of claim 1, wherein the at least one waist member comprises a plurality of eyelets coupled to the frame at locations around the outer circumference and arranged substantially perpendicular to the longitudinal axis and within a threshold distance of the outflow end of the frame, the plurality of eyelets being threaded with the control wire.

3. The implantable device of claim 2, wherein the actuation member is an elongate tube arranged substantially parallel to the longitudinal axis and at least partially encased between the first polymeric covering and the second polymeric covering, and wherein the control wire is further threaded through the elongate tube.

4. The implantable device of claim 1, wherein the first polymeric covering and the second polymeric covering are heat shrunk together around the frame at the inflow end of the frame.

5. The implantable device of claim 1, wherein the frame comprises a plurality of angled struts arranged to form two or more circumferentially extending rows of cells.

6. The implantable device of claim 5, wherein the two or more rows of cells comprise:
a first row of cells at the outflow end of the frame, wherein the cells of the first row of cells have a diamond shape and are formed of a first set of first angled struts of the plurality of angled struts, the first row of cells terminating in a plurality of outflow crowns configured to move radially inward toward the longitudinal axis of the frame and move radially outward away from the longitudinal axis in response to actuation of the control wire; and
a second row of cells upstream of the first row of cells, wherein the cells of the second row of cells have a diamond shape and are formed of a second set of second angled struts of the plurality of angled struts.

7. The implantable device of claim 6, wherein:
the first angled struts having a first length and first width;
the second angled struts having a second length and a second width; and
the first length is greater than the second length and the first width is less than the second width.

8. The implantable device of claim 6, wherein:
the two or more rows of cells further comprise a third row of cells located upstream of the second row of cells, wherein the cells of the third row of cells are formed of a third set of third angled struts of the plurality of angled struts, wherein the third row of cells terminate in a plurality of inflow crowns;
the frame further comprises a plurality of deployment members connected to and extending from respective ones of the inflow crowns, wherein the deployment members are configured to form a releasable connection with a delivery device.

9. The implantable device of claim 6, wherein the first struts have a first radial stiffness and the second struts have a second radial stiffness, the first radial stiffness being less than the second radial stiffness.

10. The implantable device of claim 6, wherein the first set of first angled struts are arranged to form a first circumferentially extending row of first angled struts at the outflow end of the frame and a second circumferentially extending row of first angled struts downstream of the first circumferentially extending row of first angled struts, wherein the first and second circumferentially extending rows of first angled struts are connected to each other by a plurality of joints, wherein the at least one waist member is located at the joints.

11. The implantable device of claim 1, wherein actuating the control wire results in configuring the implantable device in an unrestricted blood flow state or a restricted blood flow state, wherein:
the unrestricted blood flow state corresponds to the outflow end radially expanding away from the longitudinal axis of the frame to allow blood flow through the blood vessel; and
the restricted blood flow state corresponds to the outflow end radially collapsing toward the longitudinal axis of the frame to reduce blood flow through the blood vessel.

12. Then implantable device of claim 1, further comprising a skirt membrane wrapped around an exterior surface of the second polymeric covering.

13. The implantable device of claim 1, wherein the first polymeric covering and the second polymeric covering form a substantially star-shaped or polygon-shaped cross section at the outflow end of the frame when the implantable device is in an unrestricted blood flow state, and the substantially star-shaped or polygon-shaped cross section is reduced in size when the outflow end of the frame is collapsed in response to actuation of the control wire.

14. An implantable device for dynamically occluding a blood vessel, the implantable device comprising:
an annular frame having a lumen extended therethrough;
a sealing element disposed along an exterior of the frame and along an interior of the frame opposite the exterior of the frame, the sealing element being configured to seal portions of the frame between the exterior and the interior of the frame; and
an actuation member coupled to the frame and configured to alter a diameter of the frame to modulate blood flow through the lumen.

15. The implantable device of claim 14, wherein the actuation member is at least partially sealed within the sealing element.

16. The implantable device of claim 14, wherein the actuation member is configured to alter the diameter of the frame responsive to receiving a signal from a pressure sensor coupled to the frame.

17. The implantable device of claim 16, wherein the pressure sensor is coupled to a processor adapted to improve heart function by causing actuation of the actuation member responsive to receiving the signal from the pressure sensor.

18. The implantable device of claim 14, further comprising:
a waist member disposed along a portion of the frame and within the sealing element, the waist member being configured to flexibly collapse an outflow end of the frame or expand the outflow end of the frame, in response to actuation of a control wire coupled to the waist member and the actuation member.

19. The implantable device of claim 18, wherein:
the actuation member is controlled by a power source and coupled to the control wire;
the actuation member is configured to send a first signal to the control wire to activate application of tension to the control wire; and
the actuation member is configured to send a second signal to the control wire to activate release of the tension from the control wire.

20. A method of treatment for reducing blood flow at a target site in a blood vessel of a subject, the method comprising:
implanting a device in the blood vessel, the device comprising:
a frame having an inflow end, an actuatable outflow end, and a longitudinal axis extending therethrough;
a first polymeric covering disposed on an inner surface of the frame;
second polymeric disposed on an outer surface of the frame, wherein the first polymeric covering is coupled to portions of the second polymeric covering to form a circumferentially sealed space defined to extend around a portion of a circumference of the outer surface of the frame; and
a skirt membrane wrapped around an exterior surface of the second polymeric covering; and
actuating the outflow end of the device to modulate a flow of blood within the blood vessel.
